SEQUENCE LISTING (1 sequence)

SEQ ID NO: 1 - protein, 10 aa, Artificial Sequence
  Sythetically generated peptide
EDVVXCSMSY

(19) United States
(12) Patent Application Publication
(10) Pub. No.: US 2010/0189688 A1
(43) Pub. Date: Jul. 29, 2010

(54) Dose forms comprising VX-950 and their dosage regimen

(75) Inventors: Lindsay McNair, Allston, MA (US); Robert S. Kauffman, Chestnut Hill, MA (US); John J. Alam, Cambridge, MA (US); Bambang S. Adiwijaya, Belmont, MA (US); Varun Garg, Framingham, MA (US); Ann D. Kwong, Cambridge, MA (US); Tara L. Kieffer, Brookline, MA (US)

Correspondence Address: Jonathan P. O''Brien, Ph.D.;Honigman Miller Schwartz & Cohn LLP, 350 East Michigan Avenue, Suite 300, Kalamazoo, MI, 49007, US

(73) Assignee: VERTEX PHARMACEUTICALS INCORPORATED, Cambridge, MA (US)

(21) Appl. No.: 12/592,225

(22) Filed: Nov. 20, 2009

Related U.S. Application Data
(63) Continuation of application No. PCT/US08/06572, filed on May 21, 2008.
(60) Provisional application No. 60/994,430, filed on Sep. 19, 2007.
(60) Provisional application No. 60/931,108, filed on May 21, 2007.

(51) Int. Cl. A61K 38/21

(52) U.S. Cl. 424/85.7; 424/85.4

(57) ABSTRACT

The present invention relates to antiviral therapies and compositions for treating or preventing Hepatitis C infections in patients and relates to other methods disclosed herein. The invention also relates to kits and pharmaceutical packs comprising compositions and dosage forms. The invention also relates to processes for preparing these compositions, dosages, kits, and packs.

### CROSS-REFERENCE

The present application claims priority to U.S. Application No. 60/931,108, filed on May 21, 2008, and U.S. Application No. 60/994,430, filed on Sep. 19, 2008, the contents of which are incorporated herein by reference in their entireties.

### TECHNICAL FIELD OF THE INVENTION

The present invention relates to methods for treating Hepatitis C virus infections.

### BACKGROUND OF THE INVENTION

Infection by Hepatitis virus (“HCV”) is a compelling human medical problem. HCV is recognized as the causative agent for most cases of non-A, non-B hepatitis, with an estimated human sero-prevalence of 3% globally (see, e.g., A. Alberti et al., “Natural History of Hepatitis C,” J. Hepatology, 31 (Suppl. 1), 17-24 (1999)). Nearly four million individuals may be infected in the United States alone (see, e.g., M. J. Alter et al., “The Epidemiology of Viral Hepatitis in the United States, Gastroenterol. Clin. North Am., 23, 437-455 (1994); M. J. Alter, “Hepatitis C Virus Infection in the United States,” J. Hepatology, 31 (Suppl. 1), 88-91 (1999)).

Of persons who become infected with HCV, 20-25% may be able to clear the virus after the acute infection, but 75-80% will develop chronic Hepatitis C infection. (See, e.g., preface, Frontiers in Viral Hepatitis, Ed. R F Schinazi, J-P Sommadossi, and C M Rice, p. xi., Elsevier (2003)). This usually results in recurrent and progressively worsening liver inflammation, which often leads to more severe disease states such as cirrhosis and hepatocellular carcinoma (see, e.g., M. C. Kew, “Hepatitis C and Hepatocellular Carcinoma”, FEMS Microbiology Reviews, 14, 211-220 (1994); I. Saito et. al., “Hepatitis C Virus Infection is Associated with the Development of Hepatocellular Carcinoma,” Proc. Natl. Acad. Sci. USA, 87, 6547-6549 (1990)). Unfortunately, there are no broadly effective treatments for the debilitating progression of chronic HCV.

The HCV genome encodes a polyprotein of 3010-3033 amino acids (see, e.g., Q. L. Choo, et. al., “Genetic Organization and Diversity of the Hepatitis C Virus,” Proc. Natl. Acad. Sci. USA, 88, 2451-2455 (1991); N. Kato et al., “Molecular Cloning of the Human Hepatitis C Virus Genome From Japanese Patients with Non-A, Non-B Hepatitis,” Proc. Natl. Acad. Sci. USA, 87, 9524-9528 (1990); A. Takamizawa et al., “Structure and Organization of the Hepatitis C Virus Genome Isolated From Human Carriers,” J. Virol., 65, 1105-1113 (1991)0. The HCV nonstructural (NS) proteins are presumed to provide the essential catalytic machinery for viral replication. The NS proteins are derived by proteolytic cleavage of the polyprotein (see, e.g., R. Bartenschlager et. al., “Nonstructural Protein 3 of the Hepatitis C Virus Encodes a Serine-Type Proteinase Required for Cleavage at the NS3/4 and NS4/5 Junctions,” J. Virol., 67, 3835-3844 (1993); A. Grakoui et. al., “Characterization of the Hepatitis C Virus-Encoded Serine Proteinase: Determination of Proteinase-Dependent Polyprotein Cleavage Sites,” J. Virol., 67, 2832-2843 (1993); A. Grakoui et. al., “Expression and Identification of Hepatitis C Virus Polyprotein Cleavage Products,” J. Virol., 67, 1385-1395 (1993); L. Tomei et. al., “NS3 is a serine protease required for processing of hepatitis C virus polyprotein”, J. Virol., 67, 4017-4026 (1993)).

The HCV NS protein 3 (NS3) contains a serine protease activity that helps process the majority of the viral enzymes, and is thus considered essential for viral replication and infectivity. It is known that mutations in the yellow fever virus NS3 protease decreases viral infectivity (see, e.g., Chambers, T. J. et. al., “Evidence that the N-terminal Domain of Nonstructural Protein NS3 From Yellow Fever Virus is a Serine Protease Responsible for Site-Specific Cleavages in the Viral Polyprotein”, Proc. Natl. Acad. Sci. USA, 87, 8898-8902 (1990)). The first 181 amino acids of NS3 (residues 1027-1207 of the viral polyprotein) have been shown to contain the serine protease domain of NS3 that processes all four downstream sites of the HCV polyprotein (see, e.g., C. Lin et al., “Hepatitis C Virus NS3 Serine Proteinase: Trans-Cleavage Requirements and Processing Kinetics”, J. Virol., 68, 8147-8157 (1994)).

The HCV NS3 serine protease and its associated cofactor, NS4A, help process all of the viral enzymes, and is thus considered essential for viral replication. This processing appears to be analogous to that carried out by the human immunodeficiency virus aspartyl protease, which is also involved in viral enzyme processing. HIV protease inhibitors, which inhibit viral protein processing are potent antiviral agents in man, indicating that interrupting this stage of the viral life cycle results in therapeutically active agents. Consequently it is an attractive target for drug discovery.

There are not currently any satisfactory anti-HCV agents or treatments. Until recently, the only established therapy for HCV disease was interferon treatment. The first approved therapy for HCV infection was treatment with standard (non-pegylated) interferon-alfa. However, interferons have significant side effects (see, e.g., M. A. Wlaker et al., “Hepatitis C Virus: An Overview of Current Approaches and Progress,” DDT, 4, 518-29 (1999); D. Moradpour et al., “Current and Evolving Therapies for Hepatitis C,” Eur. J. Gastroenterol. Hepatol., 11, 1199-1202 (1999); H. L. A. Janssen et al. “Suicide Associated with Alfa-Interferon Therapy for Chronic Viral Hepatitis,” J. Hepatol., 21, 241-243 (1994); P. F. Renault et al., “Side Effects of Alpha Interferon,” Seminars in Liver Disease, 9, 273-277, (1989)) and interferon alfa monotherapy induces long term remission in only a fraction (˜25%) of cases (see, e.g., O. Weiland, “Interferon Therapy in Chronic Hepatitis C Virus Infection”, FEMS Microbiol. Rev., 14, 279-288 (1994)). The addition of ribavirin to the treatment regimen increases response rates slightly. Recent introductions of the pegylated forms of interferon (PEG-INTRON® and PEGASYS®), which has also been combined with ribavirin have resulted in only modest improvements in remission rates and only partial reductions in side effects. The current standard of care is a treatment regimen lasting 24-48 weeks, depending on prognostic factors such as HCV genotype and demonstration of initial response to therapy. Moreover, the prospects for effective anti-HCV vaccines remain uncertain.

Thus, there is a need for anti-HCV therapies and appropriate dose regimens for anti-HCV compounds.

HCV and other diseases and disorders are associated with liver damage. There is also a need for therapies and appropriate dose regimens for treating liver damage.

### SUMMARY OF THE INVENTION

The present invention provides a treatment for Hepatitis C virus infections. The invention therefore provides for the prevention of the clinical sequelae of Hepatitis C viral infections.

The present invention also provides a treatment for liver damage and liver inflammation.

### BRIEF DESCRIPTION OF THE FIGURES

FIG. 1A and FIG. 1B depict mean concentration time profiles by dose level (Example 3).

FIG. 2A to FIG. 2D depict derived pharmacokinetic parameters. The line inside the box represents the median, and the box represents the limits of the middle half of the data (Example 3).

FIG. 3 depicts the concentration (IU/mL) of HCV RNA in plasma over the duration of the 14-day study (Example 5).

FIG. 4 depicts the change in the concentration (IU/mL) of HCV RNA relative to baseline over the duration of the 14-day study (Example 5).

FIG. 5 depicts the change in the concentration (IU/mL) of HCV RNA relative to baseline over the duration of the 14-day study for individual subjects in the 750 mg q8h dose group (Example 5).

FIG. 6 depicts mean neopterin, ALT (alanine aminotransferase), and HCV RNA +/− SEM in all dose groups. The following symbols are used in FIG. 6: Changes from baseline in mean ALT levels±SEM (uppermost 4 lines with open symbols), mean plasma neopterin levels±SEM (middle 4 lines with open symbols) and mean plasma HCV RNA loads±SEM (lower 4 lines, closed symbols) are shown for all 3 dose groups and placebo. Patients were treated for 14 days with VX-950. *The transient increase in mean ALT level at day 12 in the 450 mg q8h group is an artifact (5 out of 10 samples were missing, median value 38 U/I, range 25-125 U/I) (Example 5).

FIG. 7 depicts mean neopterin values +/−SEM in all groups. Mean plasma neopterin levels±SEM pretreatment and at days 7 and 14 for all 3 dose groups and placebo. Note that decrease in mean neopterin is greatest in the 750 mg q8h dose group, with the highest pretreatment values and then the lowest mean values at day 14. In the 750 mg q8h dose group the decrease in neopterin compared to baseline and to placebo became significant at day 14 (*unpaired two-tailed T test, **Mann Whitney test). The broken horizontal line at Y=7.7 nmol/l represents the ULN (Example 5).

FIGS. 8, 9, and 10 depict that in vitro cleavage of TRIF (a TLR3 adaptor protein) by HCV NS3/4A protease is inhibited by VX-950.

FIG. 8 (toll-IL1 receptor domain containing adaptor inducing IFN-β TRIF or TICAM-1) depicts a schematic illustration of TRIF showing various protein binding domains. TRIF cleavage by HCV NS3 protease at Cys 372 results in two fragments-ΔC340 and ΔN372 (modified from Li et al., 2005, Proc. Nat'l. Acad. Sci. USA, 102, 2992-2997).

FIG. 9 depicts the kinetics of TRIF cleavage by HCV NS3 protease. The 35S methionine labeled coupled in vitro transcription/translation product of TRIF protein (as a substrate) was incubated with 6 μM of tNS3 protease for various time points ranging from 0-240 minutes, followed by SDS-PAGE. The gel was exposed to phosphorimager to quantitate the cleavage products. Quantitation of ΔN372 cleavage product is shown in the figure as a function of time.

FIG. 10 depicts NS3 protease dependent TRW cleavage and inhibition of TRIF cleavage by VX-950. The 35S methionine labeled coupled in vitro transcription/translation product of TRIF protein (as a substrate) was incubated with increasing concentration of tNS3 protease enzyme ranging from 0-4 μM either in the presence (Circles) or absence (Squares) of 10 μM VX-950, followed by SDS-PAGE and exposure to phosphorimager. Quantitation of the ΔN372 cleavage product is shown in the figure.

FIG. 11 shows phenotypic characteristics of the in vitro VX-950 resistant mutants. Increased resistance conferred by A156V/T mutations to VX-950 in the in vitro enzyme reactions (Ki) or in the 2-day replicon assay (IC₅₀) compared to the wild type protease. The ratio Kcat/Km of the mutants compared to the wild type enzymes is shown in the table (modified from Lin et al., J. Biol. Chem., 280, 36784-36791, 2005).

FIG. 12 shows cleavage of HCV 4A/B substrate by A156V/T mutants compared to the wild type (WT) NS3 protease: The 35S methionine labeled coupled in vitro transcription/translation product of inactivated HCV mutant protease fused to SEAP protein with 4A/B junction (as a substrate) in between, was incubated with various amounts of either the wild type (WT) (in squares) or A156V/T (in triangles and circles) tNS3 protease ranging from 0.008 μM to 6 μM, followed by SDS-PAGE and exposure to phosphorimager. Quantitation of the ΔN372 cleavage product is shown in the figure.

FIG. 13 shows cleavage of TRIF substrate by A156V/T mutants compared to the wild type (WT) NS3 protease. The 35S methionine labeled coupled in vitro transcription/translation product of TRIF (as a substrate), was incubated with various amounts of either the wild type (WT) (Squares) or A156V/T (Triangles and Circles) tNS3 protease ranging from 0.008 μM to 6 μM, followed by SDS-PAGE and exposure to phosphorimager. Quantitation of the ΔN372 cleavage product is shown in the figure.

FIG. 14 depicts mean HCV RNA, neopterin and ALT at baseline, day 7, and day 14 (Example 5).

FIG. 15 shows suppression of IFN-β promoter activity by HCV protease in Huh7 cells stimulated with Sendai virus. Huh7 cells were cotransfected with plasmids expressing luciferase gene under the control of IFN-β promoter either with the wild type (WT) or inactivated mutant (MT) protease, followed by Sendai virus (SeV) stimulation. The fold activation of luciferase gene compared to the Sendai virus uninduced controls are shown in this figure.

FIG. 16 shows that treatment with VX-950 was able to overcome the suppressive effect of HCV protease on the Sendai virus stimulated IFN-β promoter activity. Huh7 cells were co-transfected with plasmids expressing luciferase gene under the control of IFN-β promoter either with the wild type (WT) or inactivated mutant (MT) protease. These cells were either treated with DMSO (Control) or 10 μM VX-950. Cells were stimulated with Sendai virus (SeV) and luciferase activity was measured 16 hours post-infection. Fold activation of luciferase gene, compared to the Sendai virus uninduced controls are shown in this figure.

FIG. 17 shows that VX-950 treatment lead to decreases in HCV RNA in previous nonresponders to HCV therapy (FIG. 17 A) and treatment-naïve Patients (FIG. 17B). Median HCV RNA levels of patients in each treatment regimen are shown. Plasma HCV RNA concentrations were determined using the Roche COBAS TaqMan HCV/HPS assay.

FIG. 18 depicts phenotypic characterization of VX-950-resistant variants to various therapy regimes described herein.

FIG. 19 shows the estimation of treatment duration in the presence of wild type and resistant variants of FIG. 90.

FIG. 20 shows plot of the estimated duration of treatment assuming high effectiveness of Peg-IFN/RBV.

FIG. 21 shows plot of the estimated duration of treatment assuming low effectiveness of Peg-IFN/RBV.

FIG. 22 shows the viral relapse after 8 to 12 weeks of treatment.

FIG. 23 shows the estimated duration of treatment for SVR.

FIG. 24 shows the timeline of a study, which included 14 daily administration of placebo and Peg-IFN; VX-950; or VX-950 and Peg-IFN; followed by 48-week follow-up assessments period in which Peg-IFN and RBV were administered.

FIG. 25 shows the rapid antiviral response in subjects treated with VX-950 in a 14-day dosing period. Generally, at the completion of the dosing regimens, the HCV RNA levels in these treated subjects decreased by at least 2 log₁₀, and in some cases by at least 4 log₁₀.

FIG. 26 shows the individual HCV RNA levels in subjects treated with HCV/Peg-IFN-2a in a 14-day dosing period. Generally, at the completion of the dosing regimens, the HCV RNA levels in these treated subjects decreased by at least 3 log₁₀, and in some cases by at least 4 log₁₀.

### DETAILED DESCRIPTION OF THE INVENTION

This invention relates to specific doses and dosage regimens for administering VX-950. VX-950 (also known as Telaprevir) is a competitive, reversible peptidomimetic NS3/4A protease inhibitor with a steady state binding constant (ki*) of 7 nM. See, e.g., WO 02/018369.

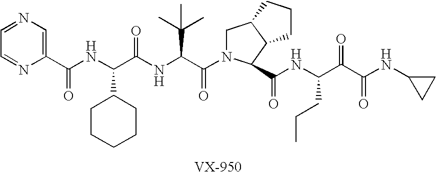

For the purpose of this invention, the compound “VX-950”, as referred to herein, includes its pharmaceutically acceptable salts, prodrugs, and solvates.

As used herein, the phrase “pharmaceutically acceptable salt(s)” of VX-950 refers to the salts of VX-950 that are safe and effective for treatment of HCV infections. Pharmaceutically acceptable salts include salts of acidic or basic groups present in VX-950. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, and pamoate salts. VX-950 may also form pharmaceutically acceptable salts with various amino acids and use of these amino acid salts is also within the scope of this invention. Suitable base salts include, but are not limited to, aluminum, calcium, lithium, magnesium, potassium, sodium, zinc, and diethanolamine salts. For a review on pharmaceutically acceptable salts, see Berge et al., J. Pharm. Sci., 66, 1-19 (1977), the contents of which are incorporated herein by reference.

As used herein, the phrase a “pharmaceutically acceptable prodrug” of VX-950 refers to a compound that may be converted under physiological conditions or by solvolysis to VX-950 or to a pharmaceutically acceptable salt of VX-950 prior to exhibiting its pharmacological effect in the treatment of HCV infections. Typically, the prodrug is formulated with the objectives of improved chemical stability, improved patient acceptance and compliance, improved bioavailability, prolonged duration of action, improved organ selectivity, improved formulation (e.g., increased hydrosolubility), or decreased side effects (e.g., toxicity). A pharmaceutically acceptable prodrug can be readily prepared from VX-950 using methods known in the art, such as those described in Burger's Medicinal Chemistry and Drug Chemistry, Vol. 1, 172-178 and 949-982, John Wiley & Sons (1995). See also Bertolini et al., J. Med. Chem., 40, 2011-2016 (1997); Shan et al., J. Pharm. Sci., 86(7), 765-767 (1997); Bagshawe, Drug Dev. Res., 34, 220-230 (1995); Bodor, Advances in Drug Res., 13, 224-331 (1984); Bundgaard, Design of Prodrugs, Elsevier Press (1985); and Larsen, Design and Application of Prodrugs, Drug Design and Development (Krogsgaard-Larsen et al., eds.), Harwood Academic Publishers (1991).

As used herein, the phrase a “pharmaceutically acceptable solvate” of VX-950 refers to a pharmaceutically acceptable solvate form of VX-950 that contains solvent molecule(s) and retains the biological effectiveness of VX-950. Examples of solvates include VX-950 in combination with water, isopropanol, ethanol, methanol, DMSO, ethyl acetate, acetic acid, or ethanolamine.

In the case of salts, prodrugs, or solvates of VX-950 that are solids, it is understood by those skilled in the art that these salts, prodrugs, and solvates may exist in different crystalline or amorphous forms, the use of all of which is also within the scope of the present invention.

VX-950 may contain one or more asymmetric carbon atoms and thus may occur as racemates and racemic mixtures, single enantiomers, diastereomeric mixtures and individual diastereomers. All such isomeric forms of these compounds are expressly included in the present invention. Each stereogenic carbon may be of the R or S configuration. The D- and L-isomers at the N-propyl side chain of VX-950 are expressly included within the scope of this invention.

VX-950 has been tested in single doses in humans and found to be well tolerated (Example 3). The incidence or severity of adverse events did not increase with VX-950 dose. No adverse events were considered to be severe (grade 3 or grade 4). The more common and severe adverse events were skin adverse events (e.g., rash and pruritus), followed by gastrointestinal events and anemia. There were no clinically significant changes from baseline laboratory values for hematology or clinical chemistry parameters. There were no clinically significant changes in physical examinations, vital signs, or electrocardiograms for any subject tested.

Applicants discovered that wild-type HCV may be eradicated by VX-950 within 10 weeks. As to VX-950-resistant rariants of HCV (with a 7-20 fold increase in IC₅₀), they may be eradicated by a follow-up of Peg-IFN/RBV dose regimen for 10-24 weeks.

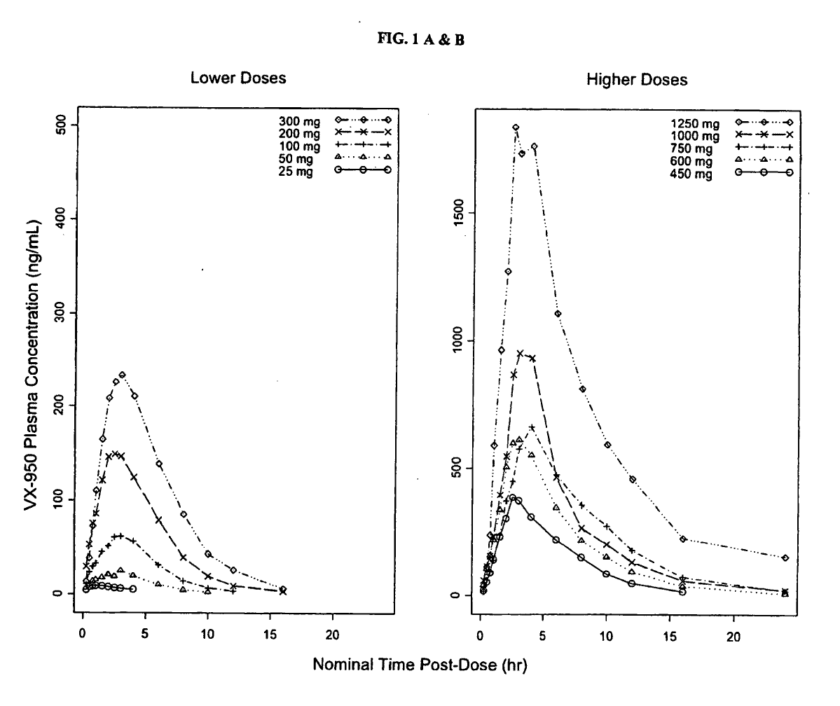

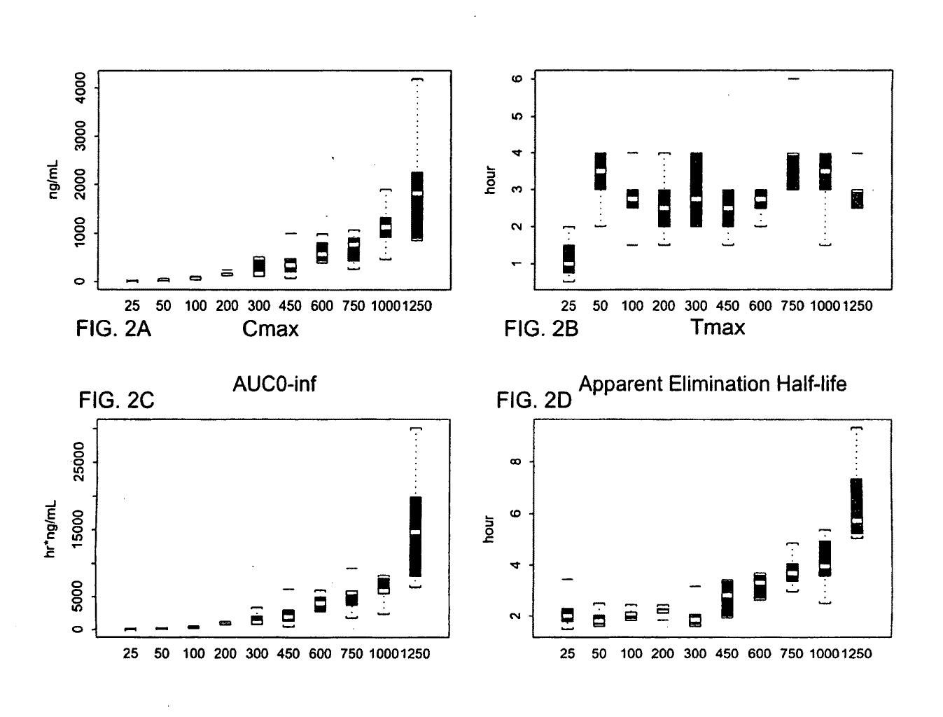

An analysis was performed to determine the pharmacokinetic profile of VX-950. The data are shown in FIG. 1 and FIG. 2.

Liver exposures to VX-950 were predicted based on the integrated preclinical and clinical data. The predicted human liver exposures were combined with results of the VX-950 replicon assay and the infectious virus assay to determine the doses that are anticipated to be well tolerated and produce therapeutic benefit. The predicted average liver concentration values are up to 57-fold of the replicon assay IC₉₀ and up to 113-fold of the replicon assay IC₅₀ in the dose range studied.

These results indicate that the dose regimen of applicants' invention will achieve liver concentrations of VX-950 substantially in excess of the IC₅₀ and IC₉₀ determined in non-clinical studies.

Accordingly, one embodiment of this invention provides pharmaceutical compositions each comprising VX-950 and a pharmaceutically acceptable carrier. The amount of VX-950 in these pharmaceutical compositions can be from about 100 mg to about 1500 mg, from about 300 mg to about 1500 mg, from about 300 mg to about 1250 mg, about 450 mg, about 750 mg, or about 1250 mg. Each of these pharmaceutical compositions can be administered, e.g., once, twice, or three times per day. Each of these compositions can be in one or more dosage forms (e.g., ampule, capsule, cream, emulsion, fluid, grain, drop, injection, suspension, tablet, powder). Each of these pharmaceutical compositions can be administered by one or more routes (e.g., orally, by infusion, by injection, topically, or parenterally) as considered appropriate by a skilled person in the art and depending on the dosage form.

Another aspect of this invention provides a method for treating or preventing HCV infections of a patient which includes administering to the patient VX-950.

In some embodiments, the amount of VX-950 is at least about 300 mg (e.g., at least about 450 mg, at least about 500 mg, at least about 750 mg, at least about 1250 mg, or at least about 1500 mg). In some embodiments, the amount of VX-950 is no more than about 1500 mg (e.g., no more than about 1250 mg, no more than about 750 mg, no more than about 450 mg, no more than about 500 mg, or no more than about 300 mg).

It should be understood that these lower and upper amounts mentioned above may be combined to provide dose ranges for administering VX-950. For example, in some embodiments, VX-950 is administered in an amount from about 300 mg to about 1250 mg or from about 300 mg to about 1500 mg.

In some embodiments, VX-950 is administered in an amount of about 450 mg, about 500 mg, about 600 mg, about 750 mg, about 1000 mg, or about 1250 mg.

In the methods of this invention, the specified amount of VX-950 can be administered, e.g., once a day, twice a day (e.g., BID; q12h), or three times a day (e.g., TID; q8h). Further, VX-950 may be administered with or without food.

VX-950 has been tested in humans and found to be effective in inhibiting HCV replication, substantially decreasing HCV RNA levels, and inhibiting the virus such that the viral RNA becomes undetectable.

Of the 8 subjects receiving 750 mg of VX-950 every 8-hours (q8h), 4 had HCV RNA levels below the limit of quantitation (LLQ 30 IU/mL) and 2 of those 4 subjects had HCV RNA levels below the limit of detection (LLD 10 IU/mL).

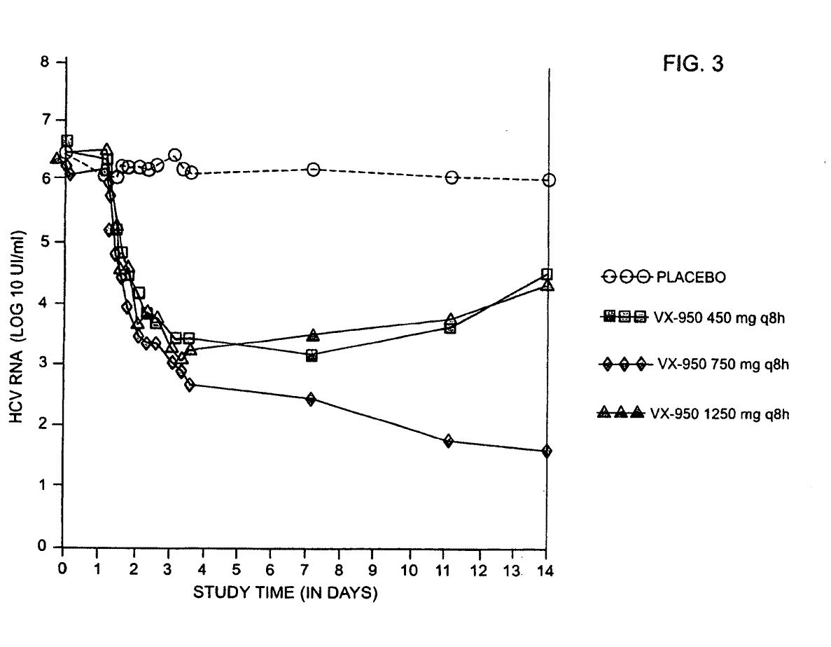

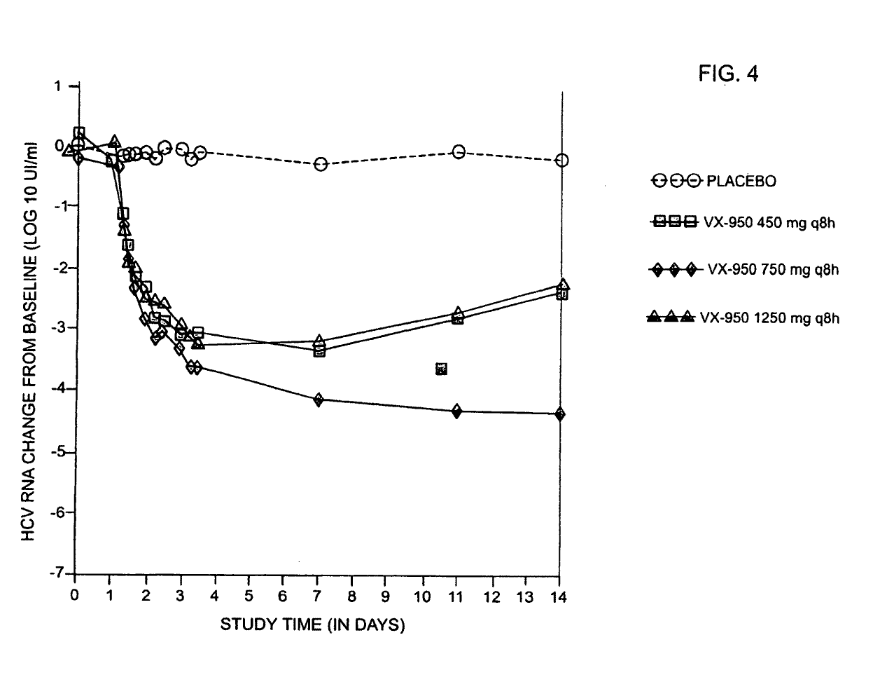

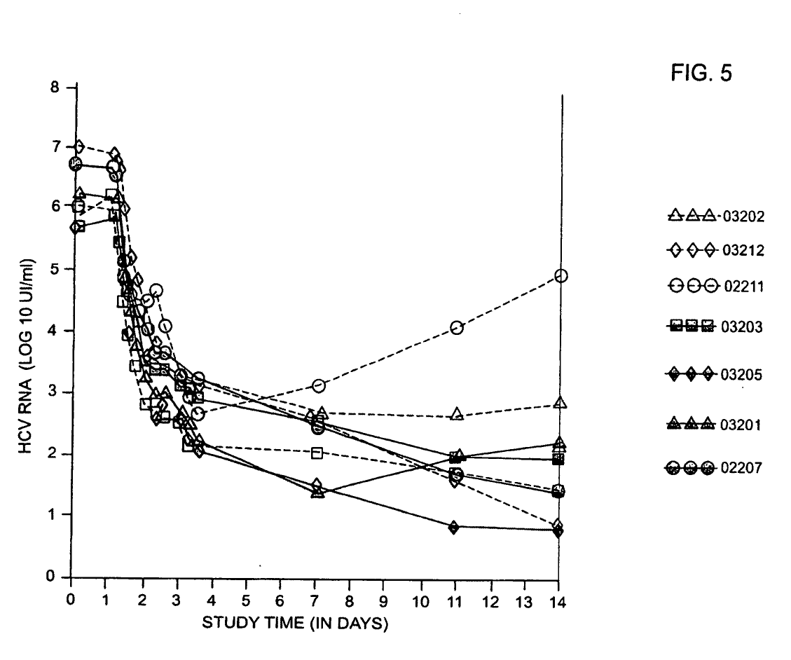

Detection of HCV RNA can be done, e.g., by using the Roche COBAS TaqMan HCV/HPS assay, available from Roche molecular Diagnostics. Subjects (or patients) receiving 750 mg of VX-950 every eight hours for 14 days achieved a median reduction in HCV-RNA of greater than 4 log₁₀ (i.e., 10,000-fold decrease) at the end of the treatment. A median reduction of HCV-RNA of greater than 2 log₁₀ was seen in each of the other two VX-950 dose groups at the end of 14 days of treatment. Every subject receiving VX-950 achieved greater than a 2 log₁₀ reduction in HCV-RNA within the first three days of treatment, and 26 of the 28 subjects receiving VX-950 had a 3 log₁₀ reduction in HCV-RNA within the first three days of treatment. See Example 5 and FIGS. 3-5.

It was demonstrated that plasma viral loads declined rapidly in patients treated with VX-950, and that there was a slow return towards baseline HCV RNA levels after the end of dosing. Specifically, the rate of return to HCV RNA baseline levels following the end of treatment was slower than the rate of decline of HCV RNA upon treatment. These results, together with achieving undetectable HCV RNA levels, indicate the effectiveness of VX-950 as a monotherapy.

Accordingly, this invention provides a method for treating a patient infected with HCV, which includes administering to the patient VX-950 (a) in an amount of about 450 mg each time, 3 times per day, once every 8 hours; (b) in an amount of about 750 mg each administration, 3 times per day, once every 8 hours; (c) in an amount of about 1250 mg each administration, 2 times per day, once every 12 hours; or (d) in an amount of about 1250 mg each time, 3 times per day, once every 8 hours.

Another aspect of this invention provides a method for treating a patient infected with HCV by administering VX-950 such that the level of HCV RNA in the patient after the administration is at least about 2 log₁₀ (e.g., at least about 4 log₁₀) lower than that before the treatment.

Yet still another aspect of this invention provides a method for administering treating a patient infected with HCV by administering VX-950 such that the level of viral RNA in the patient decreases to an undetectable level and remains at that undetectable level until a “sustained viral response” is achieved. As is used herein, the term “sustained viral response” refers to viral RNA levels remain undetectable 24 weeks after dosing is completed (or the end of VX-950 administration).

A method of this invention that employs 750 mg of VX 950 every 8 hours effectively results in higher trough levels. As used herein, the term “trough level” refers to the concentration of a drug in plasma just before the next dose, or the minimum drug concentration between two doses. It is important, particularly in viral diseases, to maintain drug levels above a certain concentration to maintain appropriate inhibition of viral replication. Advantageously, it was discovered that the dose regimen of about 750 mg of VX-950 each time, three times a day, once every 8 hours, led to the highest trough levels of VX-950.

Accordingly, in a preferred embodiment, this invention provides a method for administering VX-950 to a patient in need thereof, which includes administering the compound in an amount of about 750 mg each time, 3 times per day, once every 8 hours.

As would be recognized, it advantageous to have flexible dosing schedules. Accordingly, in another embodiment of this invention, the administration is 3 times per day, but not every 8 hours, optionally with meals. In certain embodiments, VX-950 is administered with food.

This invention also provides a method for providing VX-950 to a patient in need thereof, which includes administering to the patient an oral dose of a composition comprising VX-950, wherein said dose provides to the patient an average plasma concentration (C_{avg}) of VX-950 of at least about 750 ng/mL after the administration. In some embodiments, the C_{avg} of VX-950 is about 1000 ng/mL or about 1250 ng/mL. In some embodiments, said dose essentially contains about 750 mg of VX-950. In some embodiments, the C_{avg} is obtained or attained within 3 hours (e.g., 2 hours or 1 hour) after administration of VX-950. In some embodiments, the C_{avg} of VX-950 is maintained over about 24 hours (e.g., 5 weeks or 12 weeks).

In another aspect, this invention provides a method for treating HCV infection in a patient by administering at least one dosage form comprising VX-950 over a 24-hour period, so as to maintain a trough plasma VX-950 level minimum of about 750 ng/mL over the 24-hour period.

In some embodiments, the dosage form is administered to maintain a trough plasma VX-950 level minimum of about 800 ng/mL (e.g., about 900 ng/mL or about 1000 ng/mL) over the 24 hour period.

In certain preferred embodiments a therapeutically effective plasma concentration is obtained and a certain trough level is maintained. These methods are particularly useful for treating a human suffering from HCV infection by administering a VX-950 formulation, wherein the trough VX-950 plasma level is maintained at a minimum of about 750, 800, 900, or 1000 ng/mL over a 24 hour period. Without being bound by theory, trough levels of more than about 1500 ng/mL are thought to be not required by this invention. Accordingly, trough levels of about 750, 800, 900, 1000 ng/mL to about 1500 ng/mL (particularly 1000 to about 1500) are within the scope of this invention.

Also provided is a dosage form for delivering VX-950 to a human, wherein the dosage form comprises VX-950, said dosage form when administered at least once during a 24 hour period maintains a trough plasma VX-950 level that is at least about 750 ng/mL, 800 ng/mL, 900 ng/mL, or 1000 ng/mL over the 24 hour period to about 1500 ng/mL (particularly 1000 ng/mL to about 1500 ng/mL) over the 24 hour period.

Ideally, when a method of this invention involves treating a patient infected with HCV, the method involves achieving, relatively rapidly, a therapeutically effective plasma concentration of VX-950 and then maintaining the trough level such that an effective therapeutic response is achieved. An effective therapeutic response is, preferably, one or both of a) achieving a sustained viral response; and b) achieving undetectable HCV RNA in the plasma by at least 12 weeks (12 weeks or more). As used herein, HCV RNA being “undetectable” means that the HCV RNA is present in less than 10 IU/mL as determined by assays currently commercially available, and preferably as determined by the Roche COBAS TaqMan™ HCV/HPS assay.

The relatively rapid drop in plasma concentration may be obtained by administering a loading dose to a patient. In one embodiment, the loading dose is about 1250 mg of VX-950.

In certain dosage forms of this invention, the dosage form (other than the dosage form used to administer the loading dose) contains about 750 mg of VX-950 and the dosage form is administered once every 8 hours (i.e., q8h).

In certain embodiments, the VX-950 dosage form is administered once every 8 hours.

In certain embodiments, the treatment duration with VX-950 is shorter than the current standard of care.

In certain embodiments, VX-950 is administered for less than about 12 weeks (or less than 12 weeks).

In certain embodiments, VX-950 is administered for about 8-12 weeks (or 8-12 weeks).

In certain embodiments, VX-950 is administered for about 10 weeks (or 10 weeks).

As shown in FIGS. 90-93, modeling data indicate that administration with VX-950 may eradicate wild-type virus within 10 weeks.

In certain embodiments, VX-950 is administered for less than about 10 weeks.

In certain embodiments, VX-950 is administered for about 2 weeks.

Applicants have demonstrated that SVR was achieved in a patient receiving a 2 week treatment of VX-950.

In other embodiments, VX-950 is administered for less than about 8 weeks (or about 8 weeks or 8 weeks), less than about 6 weeks (or about 6 weeks or 6 weeks), or less than about 4 weeks (or about 4 weeks or 4 weeks).

In certain embodiments, a method according to this invention involves the treatment of a patient infected with genotype 1 Hepatitis C virus. Genotype 1 HCV infection is the most difficult strain of HCV to treat and the most prevalent strain in the United States.

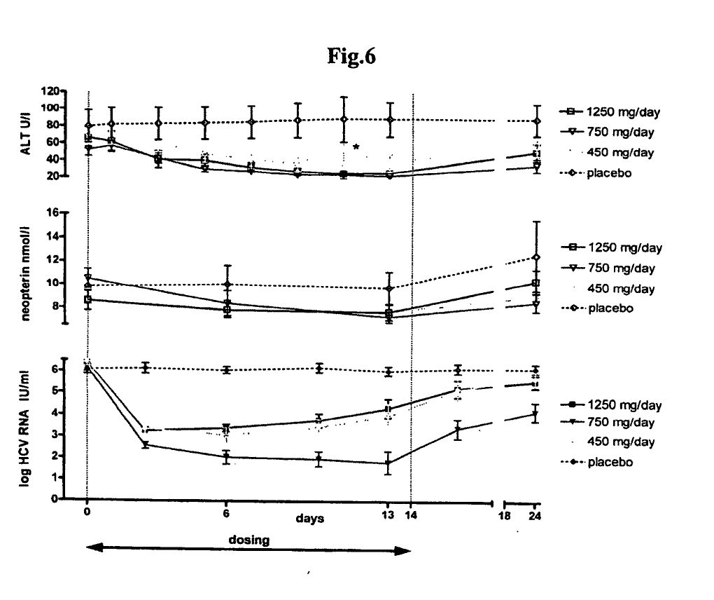

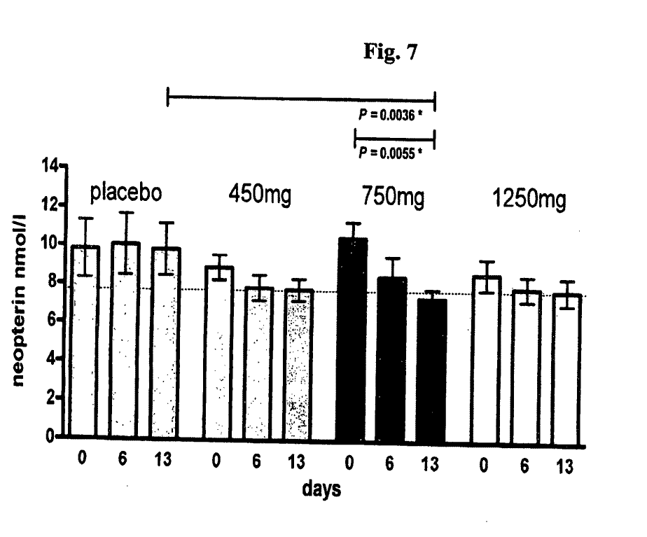

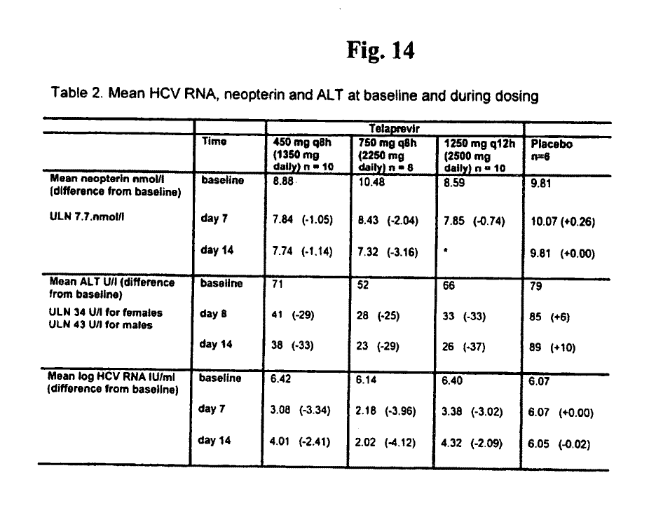

Applicants have also demonstrated that administration of VX-950 decreases neopterin and ALT levels in vivo (see FIG. 6, FIG. 7, and FIG. 14). AST (aspartate aminotransferase) levels were also decreased upon administration of VX-950. ALT is an enzyme that is present in liver cells; when liver cells are damaged or inflamed, ALT leaks from the cell into the blood. Blood ALT levels are useful as a marker of liver inflammation or damage. See, Tatyana Yashina & J. Sanders Sevall, “Hepatitis C Virus” in Use and Interpretation of Laboratory Tests in Gastroenterology, James B. Peter, ed., p. 127, (1998); and Andres T. Blei, “Liver and Biliary Tract” in Laboratory Medicine, D. A. Noe and Robert C. Rock, eds., Ch. 19, p. 363 (1994).

Neopterin (6-d-erythro-trihydroxypropylpteridine) is a pteridine derivative that is produced during the metabolism of guanosine triphosphate (GTP). Neopterin is produced primarily by monocytes and macrophages upon activation by interferon gamma or interferon alfa and is a marker of inflammation. Neopterin levels are frequently elevated in chronic HCV infection (Quiroga et al., Dig Dis Sci., 39(11): 2485-2496, 1994). The expected plasma level of neopterin in healthy individuals is between 3.1 and 7.7 nmol/L.

Accordingly, applicants determined the changes in serum neopterin concentration as a marker of monocyte/macrophage activity during administration of an inhibitor of (HCV) NS3•4A protease. As described herein, VX-950 was administered for 14 days in a randomized, double blind, placebo controlled, multiple-dose study in 34 patients infected with HCV genotype 1 (Table 1). Patients received VX-950 450 mg q8h (n=10), 750 mg q8h (n=8), 1250 mg q12h (n=10), or placebo (n=6). Serum neopterin concentrations can be measured by a quantitative competitive ELISA (ELltest® Neopterin, Brahms, Hennigsdorf, Germany) at pretreatment, at day 7 and 14, and at day 10 of follow-up. The lower limit of detection (LLD) was 2 nmol/L. HCV RNA was assessed at frequent intervals during the study by real-time PCR (COBAS® TaqMan HCV Test; linear dynamic range of 3.0×10¹ to 2.0×10⁸ HCV RNA IU/mL; LLD of 10 HCV RNA IU/mL; Roche Diagnostics, Branchburg, N.J.).

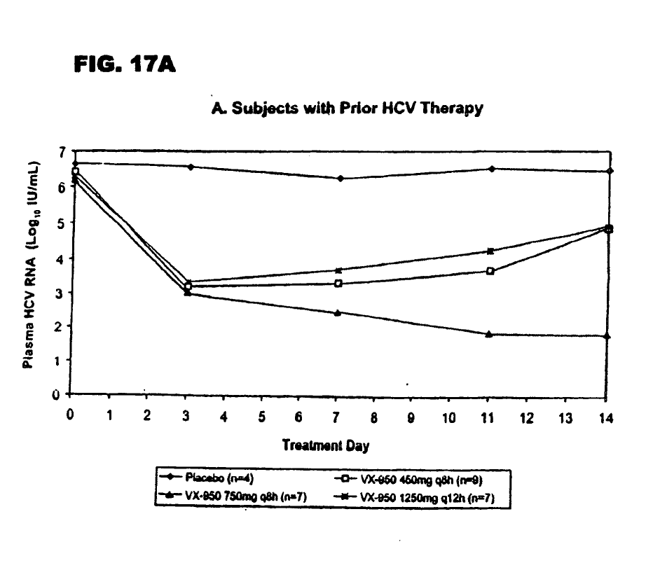

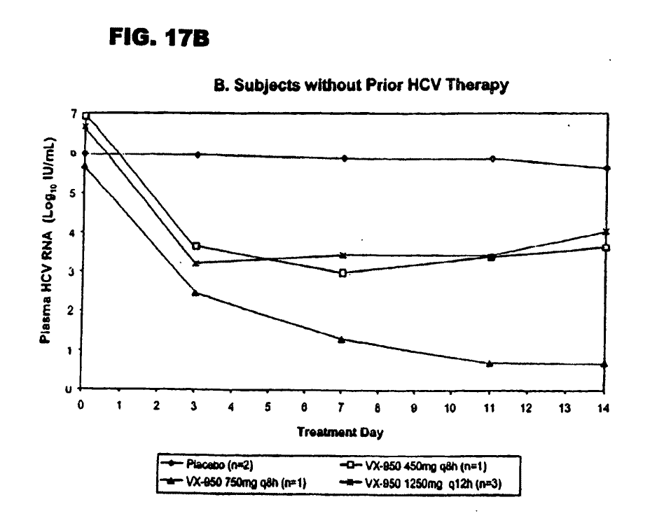

During administration of VX-950, every patient demonstrated at least 2-log₁₀ drop in viral load in all dose groups. In the 750 mg q8h dose group, mean HCV RNA dropped 3.6 log₁₀ at day 3, and 4.3 log₁₀ at day 14. In the 450 mg q8h and 1250 mg q12h dose groups, maximal effect was seen at day 3 to day 7 followed by an increase in mean viral load between day 7 and day 14. Mean viral loads increased in all dose groups during follow-up. Advantageously, both HCV treatment naïve and previously treated patients benefit from the methods of this invention. As depicted in FIG. 17A and FIG. 17B, both prior-treated patients and treatment naïve patients responded to VX-950. For the avoidance of doubt, patients that may be treated according to the methods of this invention include those where HCV treatment has not been tried or has failed, including non-responding, rebound, relapse, and breakthrough patients.

Baseline neopterin was elevated in 23/34 patients (mean 9.33 nmol/L; upper limit of normal (ULN) 7.7 nmol/l). In the 750 mg dose group the decrease in neopterin compared to baseline and to placebo became significant at day 14 (750 mg q8h dose group baseline v day 14 10.48±0.84 nmol/L v 7.32 f 0.48 nmol/L P=0.0104, Mann Whitney test; 750 mg q8h dose group v placebo day 14 7.32±0.48 nmol/l v 9.81±1.36 nmol/l P=0.0036, unpaired two-tailed T test). Mean neopterin levels were within normal values at day 14 only in the 750 mg q8h dose group (FIG. 7 and FIG. 14). In the 450 mg q8h dose group and the 1250 mg q12h dose group, decreases in mean neopterin levels were smaller (FIGS. 6, 7, and 14). Mean neopterin levels did not change in the placebo group (FIG. 6 and FIG. 7). Mean neopterin levels increased in all dose groups during follow-up.

The serum alanine aminotransferase (ALT) level can be measured using commercially available methods. Mean ALT levels, elevated at baseline, decreased during dosing in all groups (FIG. 6). Mean ALT levels increased, returned toward baseline, in all dose groups during follow up.

Although HCV RNA increased in the 450 mg dose group and 1250 mg dose group after day 7, neopterin and especially ALT continued to decrease. Changes in mean neopterin concentration correlated with decline in HCV RNA and ALT levels during dosing of VX-950. Maximal decline in mean neopterin concentration was in the 750 mg q8h dose group at day 14. This was also the dose group with maximal reductions in HCV RNA at day 14. After day 7 in the 450 mg q8h and 1250 mg q12h dose groups, ALT and neopterin levels decreased while HCV RNA levels increased. These data suggest that inhibition of HCV replication by VX-950 results in a marked decline in systemic inflammatory activity associated with viral infection.

VX-950 also ameliorates elevated ALT levels in an animal model (see WO 2005/025517). Specifically, expression of WT-HCV protease-SEAP in SCID mice results in elevated ALT levels that can be ameliorated by treatment with VX-950. Expression of WT-HCV protease alone in SCID mice also results in time and dose dependent elevation of ALT levels.

Accordingly, this invention provides a method for decreasing (including normalizing) ALT levels in a patient. The method includes administering to the patient in need thereof a therapeutically effective amount of VX-950 (e.g., about 1350 mg daily, about 2250 mg daily, or about 2500 mg daily). The patient can be infected with HCV or not infected with HCV.

In some embodiments, VX-950 is administered daily at about 450 mg or at about 750 mg every 8 hours, or at about 1250 mg every 12 hours.

Another aspect of this invention provides methods for treating or preventing one or more of liver damage, liver inflammation, steatosis, fatty liver, NAFLD, NASH, alcoholic steatosis, and Reye's syndrome in a patient that is either HCV positive or HCV negative.

Also within the scope of this invention are methods for hepatoprotection in a patient that is either HCV positive or negative.

Applicants have also demonstrated that VX-950 blocks immune evasion in vitro.

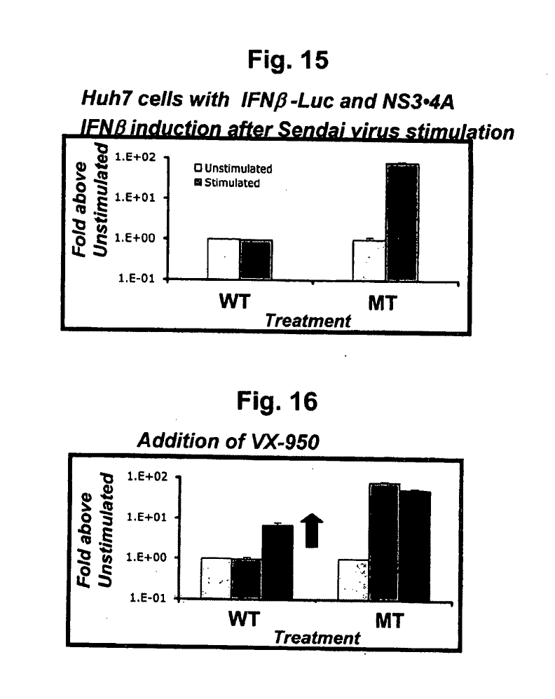

VX-950 restores IFNβ dependent gene expression in Sendai virus infected Huh7 cells. IFNβ promoter activity decreases in response to Sendai virus stimulation in the presence of WT HCVpro. VX-950 overcomes the WT HCVpro mediated suppression of IFNβ promoter activation. See FIG. 15 and FIG. 16.

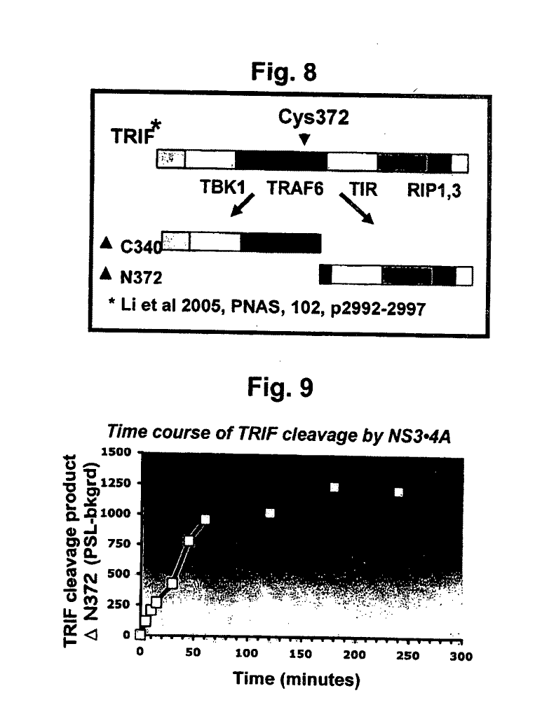

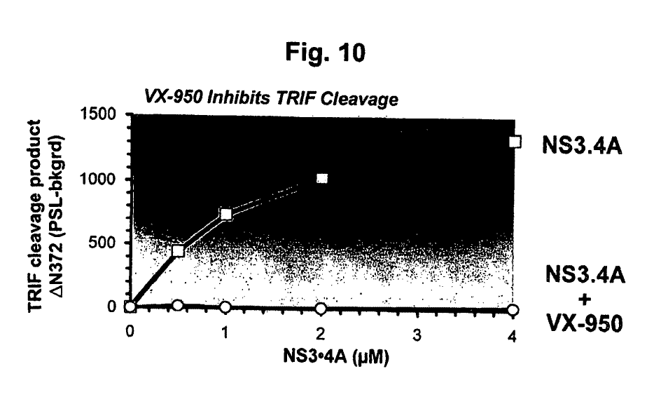

Furthermore, NS3/4A is known to be involved in evasion of innate defenses, by e.g., TRIF-dependent mechanisms (as well as viral polyprotein processing). This immune evasion leads to viral persistence. Accordingly, a compound that inhibits both viral polyprotein processing and evasion of innate defenses is desirable. Advantageously, VX-950 has been shown to do both. In particular, VX-950 inhibits in vitro cleavage of TRIF, which is a TLR3 adaptor protein. FIGS. 8-10.

Without being bound by theory, modeling suggests that VX-950 inhibits TRIF cleavage by NS3 protease. TRIF binds to non-prime side of the NS3 protease active site. VX-950 binds to the same non-prime side of the active site as TRIF and blocks TRIF cleavage.

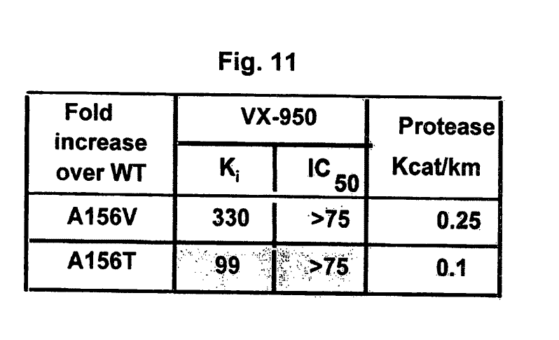

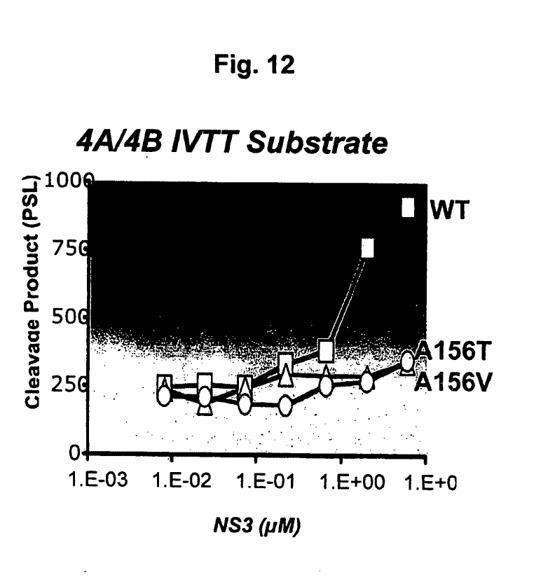

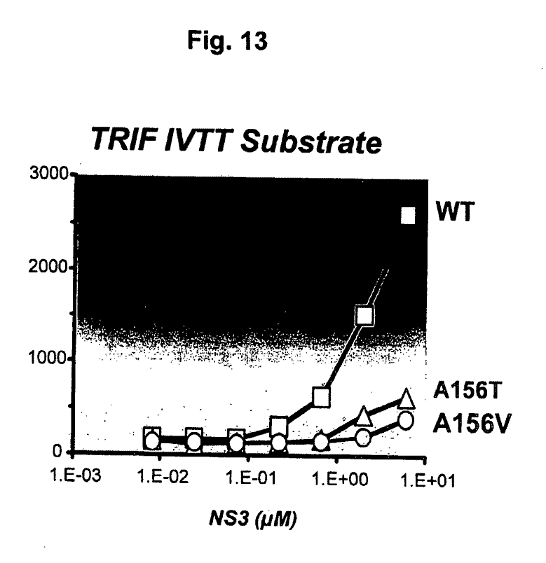

It has been shown that two VX-950 viral variants, A156T and A156V, show reduced ability to cleave either TRIF or 4A/4B (see C. Lin et al., J. Biol. Chem., (Aug. 8, 2005)). Because these viral variants are less fit, they are inefficient at both viral polyprotein processing and viral persistence. Without being bound by theory, this is related to steric hindrance of A156V affecting binding to 4A/4B and TRIF substrates. See FIGS. 11-13.

This indicates that VX-950 acts as both a direct antiviral and as an inhibitor of immune evasion. Accordingly, this invention also provides methods of inhibiting HCV protease mediated evasion of host defenses.

These results together with the in vivo data disclosed herein indicate the effectiveness of VX-950 as a monotherapy.

The amounts of VX-950 according to this invention are administered in a single dosage form or in more than one dosage form. If in separate dosage forms, each dosage form is administered about simultaneously. For the avoidance of doubt, for dosing regimens calling for dosing more than once a day, one or more pill or dose may be given at each time per day (e.g., 1 pill, three times per day or 3 pills, three times per day). Most embodiments of this invention will employ at least 2 pills per dose).

As would be realized by skilled practitioners, if a method of this invention is being used to treat a patient prophylactically, and that patient becomes infected with Hepatitis C virus, the method may then treat the infection. Therefore, one embodiment of this invention provides methods for treating or preventing a Hepatitis C infection in a patient.

In addition to treating patients infected with Hepatitis C, the methods of this invention may be used to prevent a patient from becoming infected with Hepatitis C. Accordingly, one embodiment of this invention provides a method for preventing a Hepatitis C virus infection in a patient comprising administering to the patient a composition or dosage form according to this invention.

Methods of this invention may also involve administration of another component comprising an additional agent selected from an immunomodulatory agent; an antiviral agent; an inhibitor of HCV protease (other than VX-950); an inhibitor of another target in the HCV life cycle (other than NS3/4A protease); an inhibitor of internal ribosome entry, a broad-spectrum viral inhibitor; or a cytochrome P-450 inhibitor; or combinations thereof. The additional agent is also selected from an inhibitor of viral cellular entry.

Accordingly, in another embodiment, this invention provides a method comprising administering VX-950 and another anti-viral agent, preferably an anti-HCV agent. Such anti-viral agents include, but are not limited to, immunomodulatory agents, such as α-, β-, and γ-interferons or thymosin, pegylated derivatized interferon-α compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds described in U.S. Pat. Nos. 5,807,876, 6,498,178, 6,344,465, and 6,054,472; and PCT publications WO 97/40028, WO 98/40381, and WO 00/56331; and mycophenolic acid and derivatives thereof, and including, but not limited to, VX-497, VX-148, and VX-944); or any of their combinations.

Other agents (e.g., non-immunomodulatory or immunomodulatory compounds) may be used in combination with a compound of this invention include, but are not limited to, those specified in WO 02/18369, which is incorporated herein by reference (see, e.g., page 273, lines 9-22 and page 274, line 4 to page 276, line 11 this disclosure being specifically incorporated herein by reference).

Still other agents include those described in various published U.S. Patent Applications. These publications provide additional teachings of compounds and methods that could be used in combination with VX-950 in the methods of this invention, particularly for the treatment of hepatitis. It is contemplated that any such methods and compositions may be used in combination with the methods and compositions of the present invention. For brevity, the disclosure the disclosures from those publications is referred to be reference to the publication number but it should be noted that the disclosure of the compounds in particular is specifically incorporated herein by reference. Examples of such publications include U.S. Patent Application Publication Nos.: US 20040058982, US 20050192212, US 20050080005, US 20050062522, US 20050020503, US 20040229818, US 20040229817, US 20040224900, US 20040186125, US 20040171626, US 20040110747, US 20040072788, US 20040067901, US 20030191067, US 20030187018, US 20030186895, US 20030181363, US 20020147160, US 20040082574, US 20050192212, US 20050187192, US 20050187165, US 20050049220, and US 20050222236.

Still other agents include, but are not limited to, Albuferon™ (albumin-Interferon alpha) available from Human Genome Sciences; PEG-INTRON® (peginterferon alfa-2b, available from Schering Corporation, Kenilworth, N.J.); INTRON-A®, (VIRAFERON®, interferon alfa-2b available from Schering Corporation, Kenilworth, N.J.); ribavirin (1-beta-D-ribofuranosyl-1H-1,2,4-triazole-3-carboxamide, available from ICN Pharmaceuticals, Inc., Costa Mesa, Calif.; described in the Merck Index, entry 8365, Twelfth Edition); REBETROL®(Schering Corporation, Kenilworth, N.J.); COPEGUS® (Hoffmann-La Roche, Nutley, N.J.); PEGASYS® (peginterferon alfa-2a available Hoffmann-La Roche, Nutley, N.J.); ROFERON® (recombinant interferon alfa-2a available from Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); WELLFERON® (interferon alpha n1 available from Glaxo Wellcome Ltd., Great Britain); ALFERON® (a mixture of natural alpha interferons made by Interferon Sciences, and available from Purdue Frederick Co., CT); α-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha (Reddy, K. R. et al., “Efficacy and Safety of Pegylated (40-kd) Interferon alpha-2a Compared with Interferon alpha-2a in Noncirrhotic Patients with Chronic Hepatitis C,” Hepatology, 33, 433-438 (2001); consensus interferon (INFERGEN®)(Kao, J. H., et al., “Efficacy of Consensus Interferon in the Treatment of Chronic Hepatitis,” J. Gastroenterol. Hepatol., 15, 1418-1423 (2000); lymphoblastoid or “natural” interferon; interferon tau (Clayette, P. et al., “IFN-tau, A New Interferon Type I with Antiretroviral activity” Pathol. Biol. (Paris) 47, 553-559 (1999); interleukin-2 (Davis, G. L. et al., “Future Options for the Management of Hepatitis C.” Seminars in Liver Disease, 19, 103-112 (1999); Interleukin-6 (Davis et al., “Future Options for the Management of Hepatitis C,” Seminars in Liver Disease. 19, 103-112 (1999); interleukin-12 (Davis, G. L. et al., “Future Options for the Management of Hepatitis C.” Seminars in Liver Disease, 19, 103-112 (1999); and compounds that enhance the development of type 1 helper T cell response (Davis et al., “Future Options for the Management of Hepatitis C,” Seminars in Liver Disease, 19, 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod,” J. Am. Acad. Dermatol., 43 S6-11 (2000). See also, WO 02/18369, particularly page 272, line 15 to page 273, line 8, this disclosure being specifically incorporated herein by reference.

As is recognized by skilled practitioners, VX-950 is preferably administered orally. Interferon is not typically administered orally, although orally administered forms are in development. Nevertheless, nothing herein limits the methods or combinations of this invention to any specific dosage forms or regime. Thus, each component of a combination according to this invention may be administered separately, together, or in any combination thereof. As recognized by skilled practitioners, dosages of interferon are typically measured in IU (e.g., about 4 million IU to about 12 million IU). Interferon may also be dosed by micrograms. For example, a standard dose of Peg-Intron is 1.0-1.5 μg/kg/wk and of Pegasys is 180 μg/wk.

In some aspects, the method includes the administration of agents over two phases, an initial phase and a secondary phase. For instance the initial phase can be a period of less than about 12 or 24 weeks and the secondary phase can be greater or equal to about 12 weeks, e.g., the secondary phase can be between about 12-36 weeks. In certain embodiments, the secondary phase is 12 weeks. In still other embodiments, the secondary phase is 36 weeks. In certain embodiments, the sum of the initial and secondary phase is about 24 to 48 weeks (such as 24, 36, or 48 weeks). In some embodiments, the initial and secondary phases can be identical in duration.

VX-950 may be administered in either the initial, secondary, or both phases. In some embodiments, VX-950 is administered only in the initial phase. When VX-950 is administered only in the initial phase, VX-950 may be administered alone or in combination with other agents and one or more agents are administered in the secondary phase. The other agents can be one or more anti-viral agents, one or more other agents described herein, or combinations thereof. In some embodiments, the specific agents administered in the initial and secondary phases are identical.

In some embodiments, the method includes the administration of VX-950 for two weeks (initial phase) followed by 22 weeks of administration of a combination of Peginterferon alfa-2a (Peg-IFN) and ribavirin (RBV) (secondary phase). In other embodiments, the method includes the administration of VX-950 for two weeks (initial phase) followed by 46 weeks of administration of a combination of Peg-IFN and RBV (secondary phase).

In still other embodiments, the method includes the administration of VX-950 for two weeks in combination with Peg-IFN (initial phase) followed by 22 weeks of administration of a combination of Peg-IFN and RBV (secondary phase). In other embodiments, the method includes the administration of VX-950 for two weeks in combination with Peg-IFN (initial phase) followed by 46 weeks of administration of a combination of Peg-IFN and RBV (secondary phase).

In still other embodiments, the method includes the administration of VX-950 for two weeks in combination with Peg-IFN and RBV (initial phase) followed by 22 weeks of administration of a combination of Peg-IFN and RBV (secondary phase). In other embodiments, the method includes the administration of VX-950 for two weeks in combination with Peg-IFN and RBV (initial phase) followed by 46 weeks of administration of a combination of Peg-IFN and RBV (secondary phase).

In some embodiments, the method includes the administration of VX-950 for four weeks (initial phase) followed by 20 weeks of administration of a combination of Peg-IFN and RBV (secondary phase). In other embodiments, the method includes the administration of VX-950 for four weeks (initial phase) followed by 44 weeks of administration of a combination of Peg-IFN and RBV (secondary phase).

In still further embodiments, the method includes the administration of VX-950 for four weeks in combination with Peg-IFN (initial phase) followed by 20 weeks of administration of a combination of Peg-IFN and RBV (secondary phase). In other embodiments, the method includes the administration of VX-950 for four weeks in combination with Peg-IFN (initial phase) followed by 44 weeks of administration of a combination of Peg-IFN and RBV (secondary phase).

In still other embodiments, the method includes the administration of VX-950 for four weeks in combination with Peg-IFN and RBV (initial phase) followed by 20 weeks of administration of a combination of Peg-IFN and RBV (secondary phase). In other embodiments, the method includes the administration of VX-950 for four weeks in combination with Peg-IFN and RBV (initial phase) followed by 44 weeks of administration of a combination of Peg-IFN and RBV (secondary phase).

In some embodiments, any of the initial phases described above can be conducted for about 12 weeks and the secondary phases can be conducted for about 12 weeks. Alternatively, the initial phase can be conducted for about 12 weeks and the secondary phase can be conducted for about 24 weeks. In still other aspects, the initial phase can be conducted for about 12 weeks and the secondary phase can be conducted for about 36 weeks.

In some embodiments, any of the initial phases described above can be conducted for about 8 weeks and the secondary phases can be conducted for about 16 weeks. Alternatively, the initial phase can be conducted for about 8 weeks and the secondary phase can be conducted for about 28 weeks. In still other aspects, the initial phase can be conducted for about 8 weeks and the secondary phase can be conducted for about 40 weeks.

In some embodiments, the method includes administering VX-950 in combination with Peg-IFN for less than 48 weeks. For instance, the method includes administering VX-950 in combination with Peg-IFN for less than 24 weeks.

In some embodiments, the method includes administering VX-950 in combination with Peg-IFN and RBV for less than 48 weeks. For instance, the method includes administering VX-950 in combination with Peg-IFN and RBV for less than 24 weeks.

In one embodiment, a method of this invention comprises administering VX-950 for about 2 weeks (or 2 weeks) followed by administering Peg-IFN and ribavirin for about 22 weeks (or 22 weeks) or about 46 weeks (or 46 weeks).

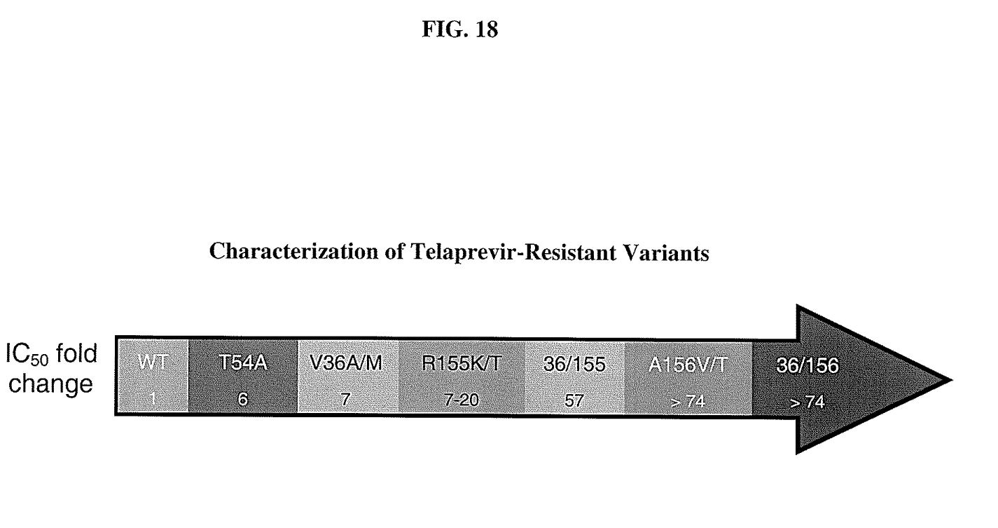

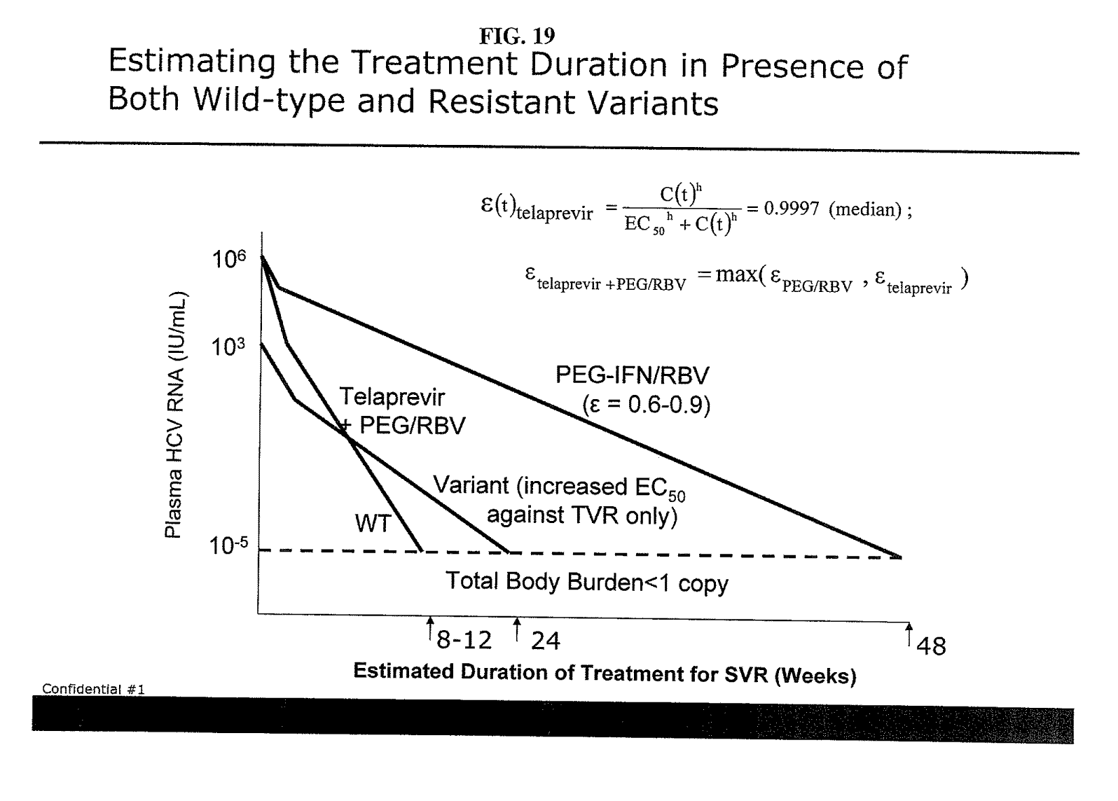

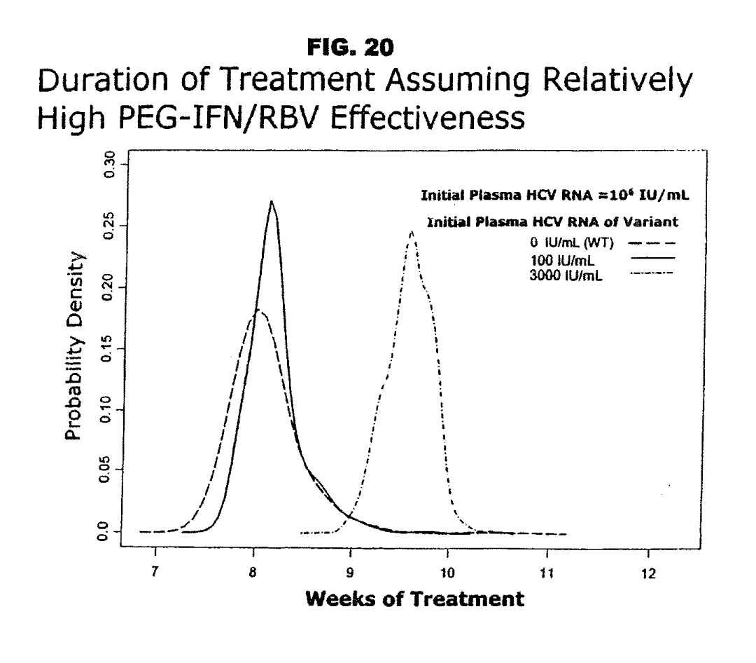

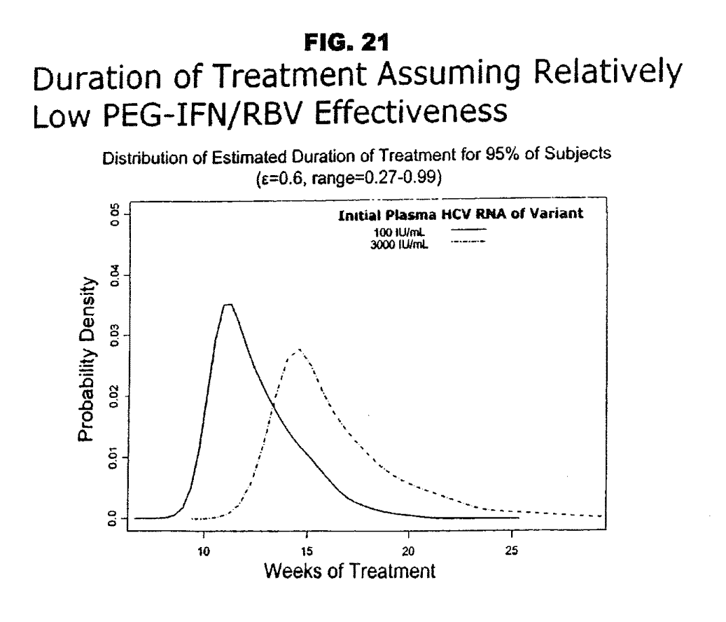

Modeling data also indicate that VX-950 resistant variants, such as V36A/M, T54A, R155K/T, A156S A156V/T, V36A/M-R155K/T, and V36A/M-A156V/T, may be eradicated mainly by administering PEG-IFN and ribavirin for about 10-24 weeks (or 8-26 weeks) following VX-950 treatment (see FIGS. 18-21). Certain of these regimens represent a reduction in treatment in the current standard of care treatment regimen lasting 24-48 weeks.

In some embodiments, the method of this invention is able to achieve week 4 RVR and week 12 undetectable status.

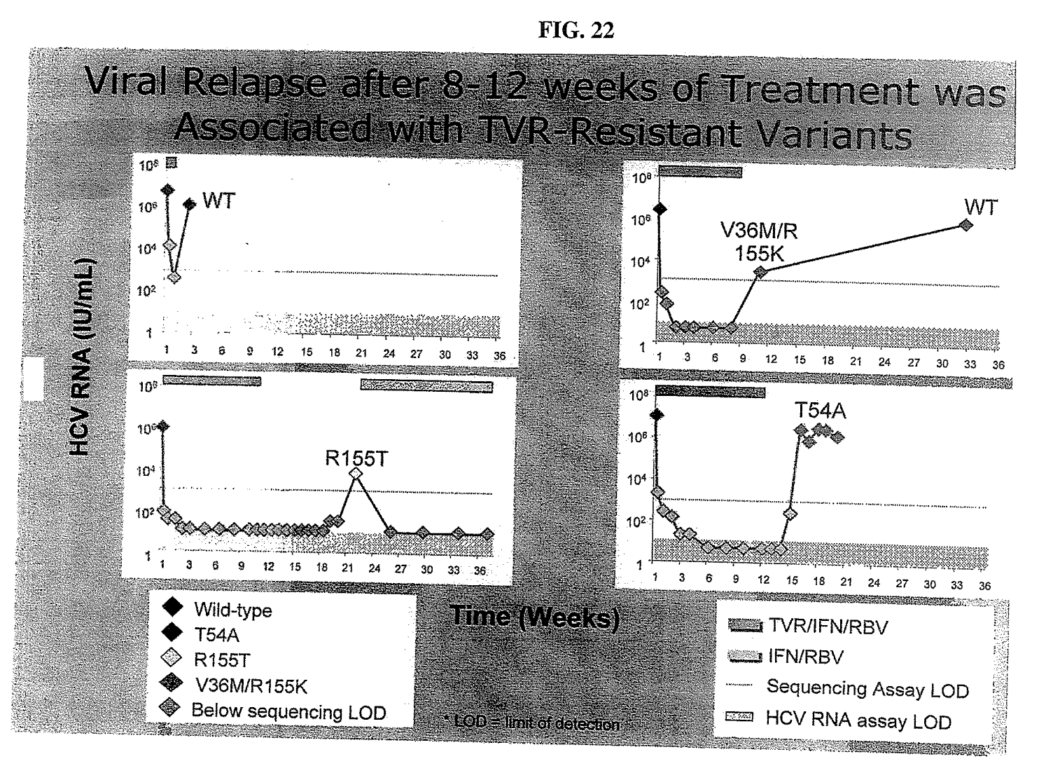

As shown in FIG. 22, the viral relapse after 8 to 12 weeks of treatment of VX-950 was associated with VX-950-resistant variants and the relapse rates with 24- or 48-week of treatment were essentially the same, particularly in subjects showing a good initial response to the treatment.

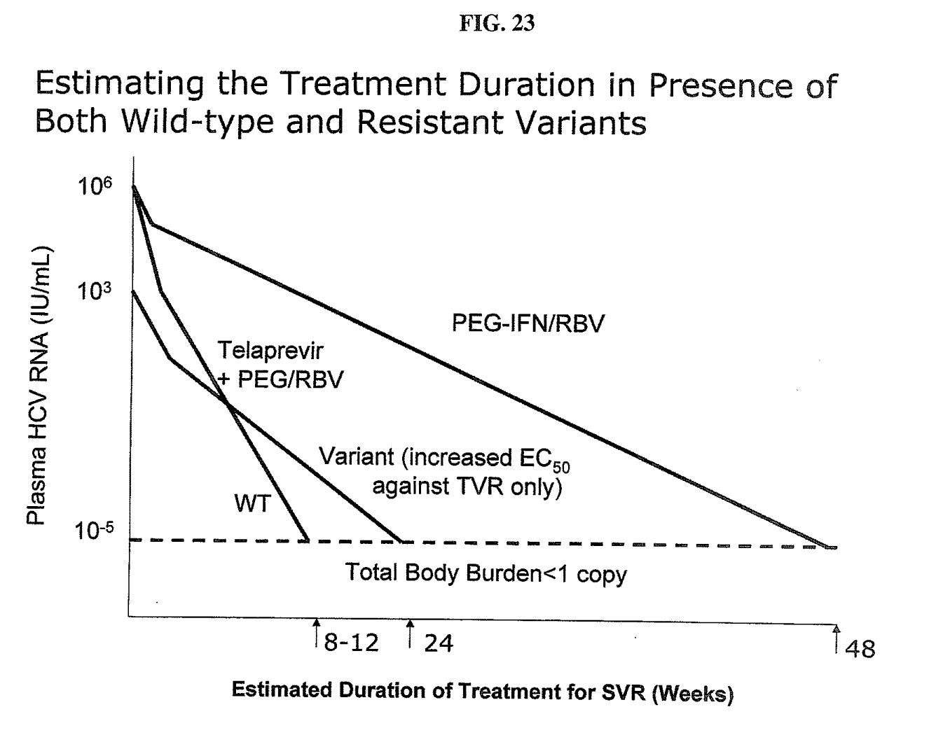

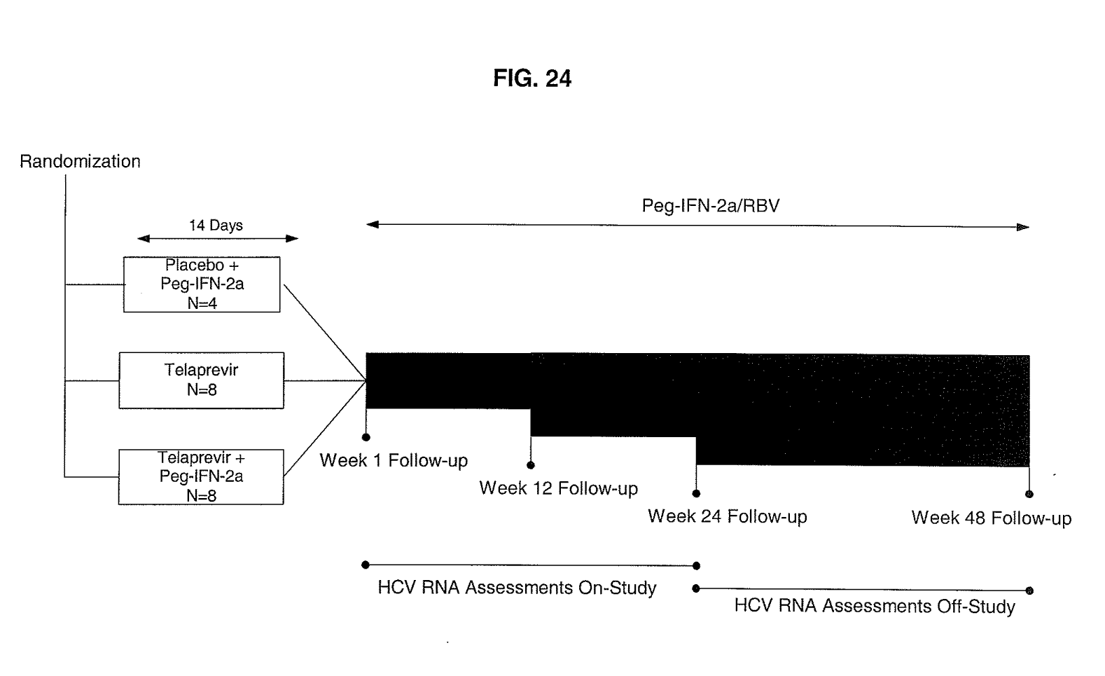

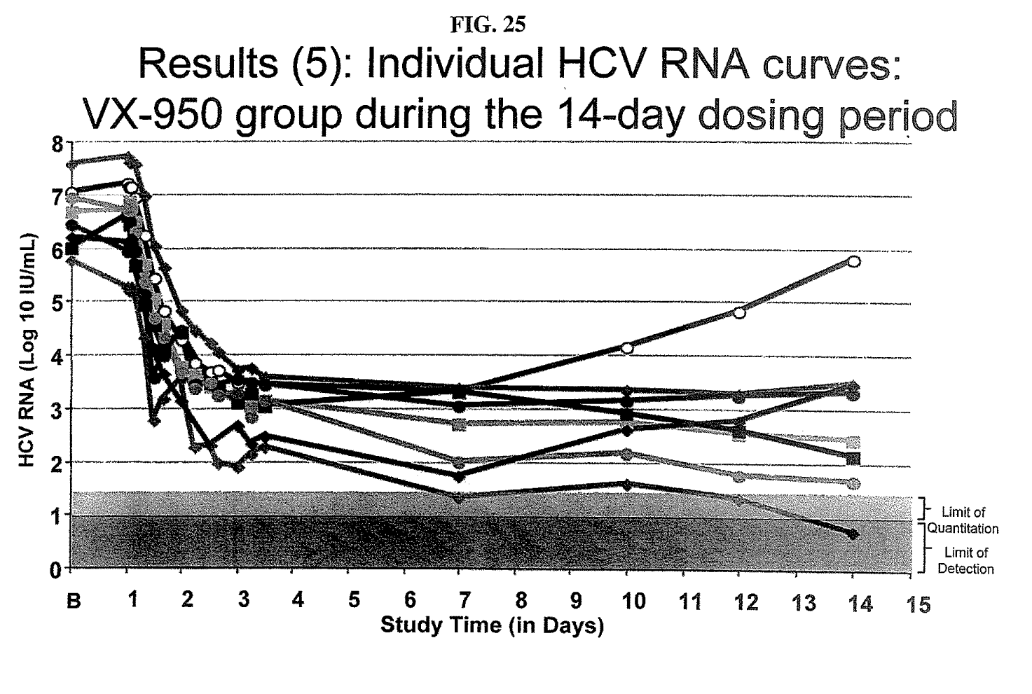

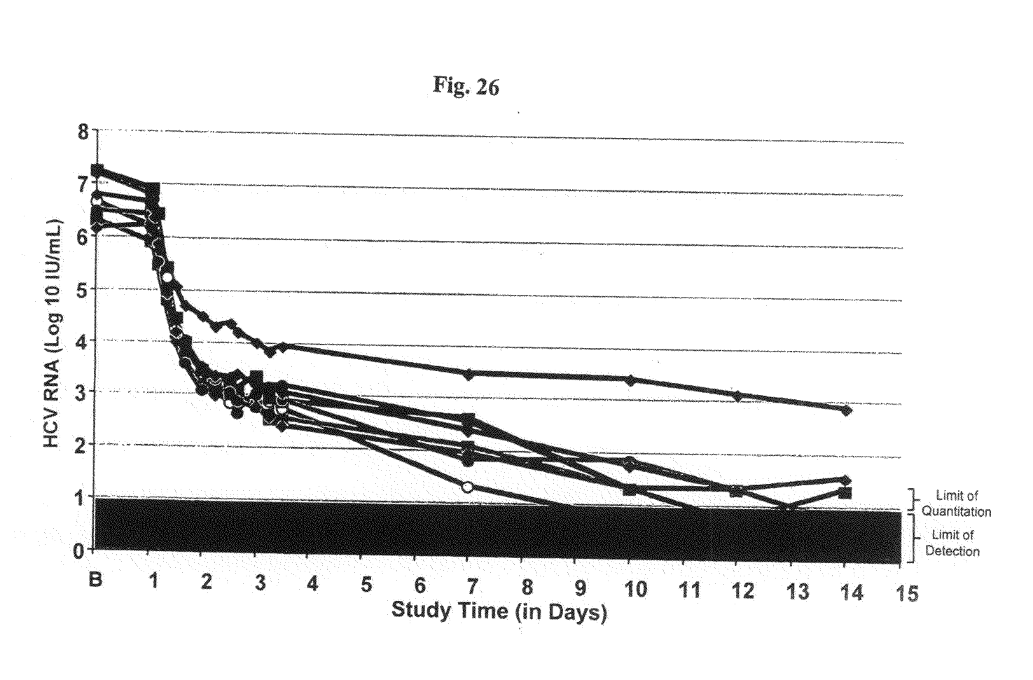

As shown in FIG. 23, the treatment with VX-950, PEG-IFN, and RBV for 12 weeks, and possibly as little as 8 weeks, appeared to be sufficient to clear wild-type virus.

Accordingly, this invention also provides methods for administering VX-950 in combination with an interferon. In certain embodiments, the interferon is administered for about 10 weeks (or 10 weeks), about 12 weeks (or 12 weeks), about 14 weeks (or 14 weeks). Ribavirin is also optionally administered for all or part of the regimen, including but not limited to, the entire regimen.

In one embodiment, a method of this invention comprises administering a combination of VX-950 and Peg-IFN for about 12 weeks (or 12 weeks).

In one embodiment, a method of this invention comprises administering a combination of VX-950 and Peg-IFN for about 12±4 weeks (e,g., 8, 12, or 16 weeks).

In one embodiment, a method of this invention comprises administering a combination of VX-950 and Peg-IFN for about 24 weeks (or 24 weeks).

In one embodiment, a method of this invention comprises administering a combination of VX-950 and Peg-IFN for about 24±4 weeks (e.g., 20, 24, or 28 weeks).

For the avoidance of doubt, it should be understood that this invention includes, but is not limited to, a regimen involving administering VX-950 and an interferon for about 8 weeks (or 8 weeks) followed by administering interferon for about 16 weeks (or 16 weeks) for a total treatment regimen of about 24 weeks (or 24 weeks). Also provided is a regimen involving administering VX-950 and an interferon for about 12 weeks (or 12 weeks) followed by administering interferon for about 12 weeks (or 12 weeks) for a total treatment regimen of about 24 weeks (or 24 weeks). Such regimens optionally provide administration of ribavirin for all or part of the regimen, including but not limited to, the entire regimen of about 24 weeks (or 24 weeks).

In one embodiment, a method of this invention comprises administering a combination of VX-950, Peg-IFN, and ribavirin for about 12 weeks (or 12 weeks).

In one embodiment, a method of this invention comprises administering a combination of VX-950, Peg-IFN, and ribavirin for about 12 weeks (or 12 weeks) followed by administering Peg-IFN and ribavirin for about 12 weeks (or 12 weeks).

In one embodiment, a method of this invention comprises administering a combination of VX-950, Peg-IFN, and ribavirin for about 12 weeks (or 12 weeks) followed by administering Peg-IFN and ribavirin for about 36 weeks (or 36 weeks).

In one embodiment, a method of this invention comprises administering a combination of VX-950, Peg-IFN, and ribavirin for about 24 weeks (or 24 weeks) followed by administering PEG-IFN and ribavirin for about 24 weeks (or 24 weeks).

In some embodiments, the method includes providing a loading dose of VX-950 (1250 mg) followed by 750 mg q8h VX-950 plus a combination of Peg-IFN and RBV.

A cytochrome P450 monooxygenase (“CYP”) inhibitor used in connection with this invention is expected to inhibit metabolism of VX-950. Therefore, the cytochrome P450 monooxygenase inhibitor would be in an amount effective to inhibit metabolism of VX-950. Accordingly, the CYP inhibitor is administered in an amount such that the bioavailability of or exposure to VX-950 is increased in comparison to VX-950 in the absence of the CYP inhibitor. CYP inhibitors include, but are not limited to, ritonavir (WO 94/14436), ketoconazole, troleandomycin, 4-methylpyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methylpyrazole, cyclosporin, and clomethiazole.

Methods for measuring the ability of a compound to inhibit cytochrome P50 monooxygenase activity are known (see, U.S. Pat. No. 6,037,157, and Yun et al., Drug Metabolism & Disposition, 21, 403-407 (1993)). Methods for evaluating the influence of co-administration of VX-950 and a CYP inhibitor in a subject are also known (US 2004/0028755). Any such methods could be used in connection with this invention to determine the pharmacokinetic impact of a combination.

One embodiment of this invention provides a method for administering an inhibitor of CYP3A4 and VX-950.

The methods herein may involve administration or co-administration of a) combinations of VX-950 and another agent; or b) VX-950 in more than one dosage form. Co-administration includes administering each inhibitor in the same dosage form or in different dosage forms. When administered in different dosage forms, the inhibitors may be administered at different times, including about simultaneously or in any time period around administration of the other dosage forms. Separate dosage forms may be administered in any order. That is, any dosage forms may be administered prior to, together with, or following the other dosage forms.

VX-950, and any additional agent, may be formulated in separate dosage forms. Alternatively, to decrease the number of dosage forms administered to a patient, VX-950, and any additional agent, may be formulated together in any combination. Any separate dosage forms may be administered at the same time or different times. It should be understood that dosage forms should be administered within a time period such that the biological effects were advantageous.

According to the regimens and dosage forms of this invention, VX-950 is present in an amount effective to decrease the viral load in a sample or in a patient, wherein said virus encodes a NS3/4A serine protease necessary for the viral life cycle (or in an amount effective to carry out a method of this invention), and a pharmaceutically acceptable carrier. Alternatively, a composition of this invention comprises an additional agent as described herein. Each component may be present in individual compositions, combination compositions, or in a single composition.

If pharmaceutically acceptable salts of compounds are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentane-propionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to a preferred embodiment, the compositions of this invention are formulated for pharmaceutical administration to a mammal, particularly a human being.

Such pharmaceutical compositions of the present invention (as well as compositions for use in methods, combinations, kits, and packs of this inventions) may be administered orally, parenterally, sublingually, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. More preferably, the compositions are administered orally.

Sterile injectable forms of the compositions of and according to this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In compositions of this invention comprising VX-950 and an additional agent, VX-950 and the additional agent should be present at dosage levels of between about 10 to 100%, and more preferably between about 10 to 80% of the dosage normally administered in a monotherapy regimen.

The pharmaceutical compositions of this invention may be orally administered in any orally acceptable dosage form including, but not limited to, capsules, tablets, pills, powders, granules, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. Acceptable liquid dosage forms include emulsions, solutions, suspensions, syrups, and elixirs.

Alternatively, the pharmaceutical compositions of this invention may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

As is recognized in the art, pharmaceutical compositions may also be administered in the form of liposomes.

Applicants have demonstrated that VX-950 is orally bioavailable. Accordingly, preferred pharmaceutical compositions of this invention are formulated for oral administration.

For the CYP inhibitor, the dosage levels of between about 0.001 to about 200 mg/kg body weight per day, would be typical. More typical would be dosage levels of between about 0.1 to about 50 mg/kg or about 1.1 to about 25 mg/kg per day.

For preferred dosage forms of ritonavir, see U.S. Pat. No. 6,037,157, and the documents cited therein: U.S. Pat. No. 5,484,801, U.S. patent application Ser. No. 08/402,690, and PCT Publications Nos. WO 95/07696 and WO 95/09614.

Administrations in connection with this invention can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, the judgment of the treating physician and the severity of the particular disease being treated, prior treatment history, co-morbidities or concomitant medications, baseline viral load, race, duration of diseases, status of liver function and degree of liver fibrosis/cirrhosis, and the goal of therapy (eliminating circulating virus per-transplant or viral eradication). The amount of active ingredients will also depend upon the particular described compound and the presence or absence and the nature of the additional anti-viral agent in the composition.

According to another embodiment, the invention provides a method for treating a patient infected with a virus characterized by a virally encoded NS3/4A serine protease that is necessary for the life cycle of the virus by administering to said patient a pharmaceutically acceptable composition of this invention. Preferably, the methods of this invention are used to treat a patient suffering from a HCV infection. Such treatment may completely eradicate the viral infection or reduce the severity thereof. Preferably, the patient is a mammal. More preferably, the patient is a human being.

The dosages herein are preferably for use in vivo. Nevertheless, this is not intended as a limitation to using of these amounts of VX-950 for any purpose. In yet another embodiment the present invention provides a method of pre-treating a biological substance intended for administration to a patient comprising the step of contacting said biological substance with a pharmaceutically acceptable composition comprising a compound of this invention. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc.

This invention also provides a process for preparing a composition comprising VX-950, or a pharmaceutically acceptable salt thereof, and a pharmaceutically acceptable carrier, adjuvant, or vehicle comprising the step of combining the VX-950, or the pharmaceutically acceptable salt thereof, and the pharmaceutically acceptable carrier, adjuvant, or vehicle, wherein the dosage of VX-950 in the composition is in accordance with any embodiment of this invention. An alternative embodiment of this invention provides a process wherein the composition comprises one or more additional agent as described herein.

This invention also provides a therapeutic regimen comprising VX-950, or a pharmaceutically acceptable salt thereof, at the dosages disclosed herein. In an alternative embodiment of this invention, the therapeutic regimen further comprises one or more of additional agent as described herein.

Pharmaceutical compositions may also be prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination of the invention by means of a single patient pack, or patient packs of each formulation, containing within a package insert instructing the patient to the correct use of the invention is a desirable additional feature of this invention.

According to a further aspect of the invention is a pack including VX-950 (in dosages according to this invention) and an information insert containing directions on the use of the combination of the invention. Any composition, dosage form, therapeutic regimen or other embodiment of this invention may be presented in a pharmaceutical pack. In an alternative embodiment of this invention, the pharmaceutical pack further comprises one or more of additional agent as described herein. The additional agent or agents may be provided in the same pack or in separate packs.

Another aspect of this involves a packaged kit for a patient to use in the treatment of HCV infection or in the prevention of HCV infection (or for use in another method of this invention), comprising: a single or a plurality of pharmaceutical formulation of each pharmaceutical component; a container housing the pharmaceutical formulation(s) during storage and prior to administration; and instructions for carrying out drug administration in a manner effective to treat or prevent HCV infection.

Accordingly, this invention provides kits for the simultaneous or sequential administration of a dose of VX-950 (and optionally an additional agent). Typically, such a kit will comprise, e.g. a composition of each compound and optional additional agent(s) in a pharmaceutically acceptable carrier (and in one or in a plurality of pharmaceutical formulations) and written instructions for the simultaneous or sequential administration.

In another embodiment, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle. The present kits will also typically include means for packaging the individual kit components, i.e., the dosage forms, the container means, and the written instructions for use. Such packaging means may take the form of a cardboard or paper box, a plastic or foil pouch, etc.

A kit according to this invention could embody any aspect of this invention such as any composition, dosage form, therapeutic regimen, or pharmaceutical pack.

The packs and kits according to this invention optionally comprise a plurality of compositions or dosage forms. Accordingly, included within this invention would be packs and kits containing one composition or more than one composition.

Although certain exemplary embodiments are depicted and described below, it will be appreciated that compounds of this invention can be prepared according to the methods described generally above using appropriate starting materials generally available to one of ordinary skill in the art.

All cited documents are incorporated herein by reference.

In order that this invention be more fully understood, the following preparative and testing examples are set forth. These examples are for the purpose of illustration only and are not to be construed as limiting the scope of the invention in any way.

### Example 1

### HCV Replicon Cell Assay Protocol

Cells containing hepatitis C virus (HCV) replicon were maintained in DMEM containing 10% fetal bovine serum (FBS), 0.25 mg per mL of G418, with appropriate supplements (“media A”).

On day 1, a replicon cell monolayer was treated with a trypsin:EDTA mixture, removed, and then media A was diluted into a final concentration of 100,000 cells per mL. 10,000 cells in 100 μL were plated into each well of a 96-well tissue culture plate, and cultured overnight in a tissue culture incubator at 37° C.

On day 2, compound VX-950 in 100% DMSO were serially diluted into DMEM containing 2% FBS, 0.5% DMSO, with appropriate supplements (“media 13”) to obtained solutions containing VX-950 at different concentrations. The final concentration of DMSO was maintained at 0.5% throughout the dilution series.

Media A on the replicon cell monolayer was removed, and then media B containing various concentrations of VX-950 was added. Media B without any compound was added to other wells as control.

Cells were incubated with VX-950 solutions in media B or control for 48 hours in a tissue culture incubator at 37° C. At the end of the 48-hour incubation period, media B was removed, and the replicon cell monolayer was washed once with PBS and stored at −80° C. prior to RNA extraction.

The culture plates with treated replicon cell monolayers were thawed, and a fixed amount of another RNA virus, Bovine Viral Diarrhea Virus (BVDV), was added to the cells in each well. RNA extraction reagents (such as reagents from RNeasy kits) were added to the cells immediately to avoid degradation of RNA. Total RNA was extracted according the instruction of manufacturer with modification to improve extraction efficiency and consistency. Finally, total cellular RNA, including HCV replicon RNA, was eluted and stored at −80° C. until further processing.

A Taqman real-time RT-PCR quantification assay was set up with two sets of specific primers and probe. One was for HCV and the other was for BVDV. Total RNA extractant from treated HCV replicon cells was added to the PCR reaction for quantification of both HCV and BVDV RNA in the same PCR well. Experimental failure was flagged and rejected based on the level of BVDV RNA in each well. The level of HCV RNA in each well was calculated according to a standard curve run in the same PCR plate. The percentage of inhibition or decrease of HCV RNA level due to VX-950 treatment was calculated using the DMSO or VX-950-free control as 0% of inhibition. The IC₅₀ (concentration at which 50% inhibition of HCV RNA level is observed) was calculated from the titration curve of any VX-950 concentrations.

The results show that VX-950 had significant inhibitory activity in the replicon assay, with the IC₅₀ of about 240 ng/mL and IC₉₀ of about 476 ng/mL.

### Example 2

### HCV Ki Assay Protocol

### HPLC Microbore Method for Separation of 5AB Substrate and Products

Substrate used this study was:

NH₂-Glu-Asp-Val-Val-(alpha)Abu-Cys-Ser-Met-Ser-Tyr-COOH. SEQ ID NO:1

A stock solution of 20 mM 5AB was made in DMSO with 0.2M DTT and stored in aliquots at −20° C. A buffer of pH 7.8 was made to contain 50 mM HEPES, 100 mM NaCl, and 20% glycerol.

Assay solutions of 100 μL were prepared according to the following table:

| | X1 (μL) | conc. in assay | |
|---|---|---|---|
| Buffer | 86.5 | See above | |
| 5 mM KK4A | 0.5 | 25 | μM |
| 1 M DTT | 0.5 | 5 | mM |
| DMSO or VX-950 | 2.5 | 2.5% | v/v |
| 50 μM tNS3 | 0.05 | 25 | nM |
| 250 μM 5AB (initiate) | 20 | 25 | μM |

The buffer, KK4A, DTT, and tNS3 were combined, and distributed 78 μL each into wells of a 96-well plate, followed by incubation at 30° C. for 5 to 10 minutes.

2.5 μL of appropriate concentration of VX-950 was dissolved in DMSO (DMSO only for control) and added to each well, followed by incubation at the room temperature for 15 minutes.

The reaction was initiated by addition of 20 μL of 250 μM 5AB substrate (25 μM concentration is equivalent to or slightly lower than the Kₘ for 5AB). The resultant mixture was incubated at 30° C. for 20 minutes, before the reaction was terminated by the addition of 25 μL of 10% TFA and the mixture transferred (in 120 μL aliquots) to HPLC vials for analysis.

SMSY product was separated from the substrate and KK4A by the following method:

Instrumentation: Agilent 1100

Degasser G1322A

Binary pump G1312A

Autosampler G1313A

Column thermostated chamber G1316A

Diode array detector G1315A

Column:

Phenomenex Jupiter; 5 micron C18; 300 angstroms; 150×2 mm; P/O 00F-4053-B0

Column thermostat: 40 C

Injection volume: 100 μL

Solvent A=HPLC grade water+0.1% TFA

Solvent B=HPLC grade acetonitrile+0.1% TFA

| Time (min) | % B | Flow (mL/min) | Max press. |
|---|---|---|---|
| 0 | 5 | 0.2 | 400 |
| 12 | 60 | 0.2 | 400 |
| 13 | 100 | 0.2 | 400 |
| 16 | 100 | 0.2 | 400 |
| 17 | 5 | 0.2 | 400 |

Stop time: 17 min
Post-run time: 10 min.

### Example 3

### Tolerance and Pharmacokinetics Studies

VX-950 was examined in a randomized, double-blind, placebo-controlled single-dose escalation study. 25 healthy male volunteers were enrolled and each received multiple single doses of VX-950 (at least 7 days apart, 3 doses of VX-950 at increasing dose levels) and 1 dose of placebo.

Doses of 25 mg to 1250 mg were evaluated. A dose escalation scheme was used that combined dose doubling and modified Fibonacci to be aggressive in the lower dose range and conservative in the higher dose range.

The results showed that VX-950 was well tolerated at all dose levels. No serious adverse events were reported during the study, and there did not appear to be an increase in adverse events with increasing dose levels.

A pharmacokinetics analysis was performed using the statistical moment approach. FIG. 1A and FIG. 1B illustrate the mean concentration-time profiles. The selected derived pharmacokinetic parameters are depicted in FIGS. 2A-2D. Pharmacokinetic analysis showed that VX-950 was absorbed with a median tₘₐₓ of 3 hours. Less than 2% of VX-950 was eliminated unchanged in the urine, indicating that the drug is primarily eliminated via the metabolic route.

### Example 4

### Infectious Virus Assay

VX-950 demonstrated an IC₅₀ of 196 ng/mL in the infectious virus assay.

### Example 5

### Effects of VX-950 Treatment

VX-950 was examined in a randomized, placebo-controlled, multiple-dose, blinded, dose escalation study in 24 healthy subjects and 36 Hepatitis C positive subjects.

The 24 healthy subjects were divided into 3 panels of 8 subjects each. In each panel, 6 subjects received VX-950 and 2 subjects received placebo. Healthy subjects were dosed with VX-950 at 450 mg, 750 mg, or 1250 mg q8h for 5 consecutive days. The healthy subjects were between the ages of 18-65 years (inclusive) and were Hepatitis B, Hepatitis C, and HIV negative. The males had a body mass index of 18.5-29.0 kg/m² (inclusive). The females had a body mass index of 18.5-32.5 kg/m² (inclusive).

Hepatitis C (genotype 1) positive subjects were divided into 3 panels of 12 subjects each for receiving VX-950 at 450 mg q8h, 750 mg q8h, or 1250 mg q12h for 14 consecutive days. In each panel, 10 subjects received VX-950 and 2 subjects received placebo. In the 750 mg group, 2 subjects withdrew prior to dosing. All other 34 subjects completed the study.

The study showed that VX-950 was well tolerated at all dose levels and no serious adverse events were reported during the study; mild and moderate adverse events were reported. Among the HCV positive subjects receiving placebo, 450 mg q8h, 750 mg q8h, and 1250 mg q12h groups, 33.2%, 10%, 12.5%, and 30%, respectively, were treatment-naïve.

The HCV positive subjects were tested post-treatment to monitor HCV RNA levels' return to baseline.

TABLE 1
SUBJECT BASELINE CHARACTERISTICS

| | | VX-950 dose | | |
|---|---|---|---|---|
| | Placebo | 450 mg q8h | 750 mg q8h | 1250 mg q12h |
| | (n = 6) | (n = 10) | (n = 8) | (n = 10) |
| Sex, n (%) | | | | |
| Male | 3 (50.0) | 8 (80.0) | 3 (37.5) | 8 (80.0) |
| Female | 3 (50.0) | 2 (20.0) | 5 (62.5) | 2 (20.0) |
| Race, n (%) | | | | |
| Caucasian | 6 (100) | 10 (100) | 8 (100) | 10 (100) |
| Age, years | | | | |
| Median | 54.0 | 47.0 | 52.0 | 43.5 |
| Range | 31-64 | 33-64 | 46-64 | 25-62 |
| BMI, kg/m² | | | | |
| Median | 24.8 | 25.8 | 27.0 | 22.2 |
| Range | 21.0-29.0 | 22.6-28.4 | 21.1-29.4 | 21.2-24.3 |
| HCV RNA, log₁₀ IU/mL | | | | |
| Mean ± SD | 6.28 ± 0.47 | 6.54 ± 0.50 | 6.18 ± 0.47 | 6.46 ± 0.41 |
| Approximate years HCV | 7.3 ± 7.6 | 9.2 ± 11.5 | 7.2 ± 7.6 | 6.9 ± 6.7 |
| infection, mean ± SD | | | | |
| HCV subtype, n (%) | | | | |
| 1* | 1 (16.7) | 0 | 2 (25.0) | 1 (10.0) |
| 1a | 2 (33.3) | 3 (30.0) | 1 (12.5) | 5 (50.0) |
| 1b | 3 (50.0) | 7 (70.0) | 5 (62.5) | 4 (40.0) |
| Prior HCV treatment | 4 (66.7) | 9 (90.0) | 7 (87.5) | 7 (70.0) |
| n (%) | | | | |

*Samples from 4 patients were classified as genotype 1 because the assay could not determine whether they were genotype 1a or 1b.
Abbreviations: BMI (body mass index); HCV (hepatitis C virus); q8h (every 8 hours); q12h (every 12 hours); SD (standard deviation).
HCV RNA change from baseline, study VX04-950-101

TABLE 2
MAXIMUM CHANGES IN HCV RNA BY CATEGORY

| | | VX-950 dose | | |
|---|---|---|---|---|
| Change From | | 450 mg | 750 mg | 1250 mg |
| Baseline in HCV | Placebo | q8h | q8h | q12h |
| RNA (log₁₀ IU/mL) | (n = 6) | (n = 10) | (n = 8) | (n = 10) |
| >−1 to <0 | 6 (100.0) | 0 | 0 | 0 |
| >−2 to ≦−1 | 0 | 0 | 0 | 0 |
| >−3 to ≦−2 | 0 | 1 (10.0) | 0 | 1 (10.0) |
| >−4 to ≦−3 | 0 | 7 (70.0) | 3 (37.5) | 9 (90.0) |
| >−5 to ≦−4 | 0 | 0 | 3 (37.5) | 0 |
| ≧−5 | 0 | 2 (20.0) | 2 (25.0) | 0 |

Values are n (%): q8h, every 8 hours; q12h, every 12 hours.

### Example 6

### Formulation of VX-950

An oral dosage formulation was prepared as follows. VX-950 and povidone K29/32 were dissolved in methylene chloride, then sodium lauryl sulfate was added to and dispersed in the VX-950 solution to form a homogenous suspension. This suspension was spray-dried using an inlet temperature of 90° C. and an outlet temperature of 56° C., and the product was collected from the cyclone. The spray-dried dispersion was fluid-bed dried at 75° C. for 8 hours. The resultant powder was pre-measured into glass vials, and just prior to dosing was suspended in water (30 mL) for administration to the subjects. In connection with dosing, each vial was washed with 3 separate portions of water, with the total volume of water being 90 mL.

VX-950 Solid Dispersion

| % (w/w) | Ingredient | |
|---|---|---|
| 49.5 | VX-950 | Spray-dried from CH₂Cl₂ |
| 49.5 | PVP K29/32 | |
| 1 | SLS | |

### Example 7

### VX-950 Validation in Human Plasma

The assay for determined VX-950 concentration in human plasma was performed by methods well known in the art. See, e.g., Wasley, A. et al., Semin. Liver Dis., 20:1-16, 2000; Alter, H. J. et al., Semin. Liver Dis., 20: 17-35, 2000; Brown, R. S. Jr. et al., Liver Transpl., 9: S10-S13, 2003; DeFrancesco, R. et al., Nature, 436(7053): 953-960, 2005; Bowen, D. G. et al., J. Hepatol., 42: 408-417, 2005; Hoofnagle, J. H., Hepatology, 36: S21-S29, 2002, Brown, R. S. Jr. et al., Nature, 436 (7053): 973-978, 2005; and Chisari, F. V., Nature, 436(7053): 930-932, 2005.

Specifically, the following VX-950 solutions were prepared and stored in capped borosilicate tubes (11.5 mL) at −20° C.:

Stock solution: 961 μg/mL of VX-950 in 2-propanol (10.0 mL)

Diluted stock solution 1: 96.1 μg/ml of VX-950 in 2-propanol (5.00 mL)

Diluted stock solution 2: 9.61 μg/ml of VX-950 in 2-propanol (10.0 mL)

Diluted stock solution 3: 0.961 μg/ml of VX-950 in 2-propanol (10.0 mL)

An internal standard stock solution was prepared to contain 1.00 mg/mL of Compound 1 (a close structural analog of VX-950) in 5.00 mL of 2-propanol, and was stored in a capped borosilicate tube (11.5 ml) at −20° C. A working solution containing the same Compound 1 was prepared to contain 300 ng/mL of Compound 1 in 100 mL of acetonitrile, and stored in a capped borosilicate bottle (100 mL) −20° C.

Sample Preparation: 100 μL of plasma and 100 μL of internal standard working solution (or acetonitrile for blank samples) were added to an extraction tube. After vortex mixing for 30 seconds, 500 μL of toluene was added and extraction was performed by vortex mixing for 30 seconds. After centrifugation at 3000 rpm at 4° C. for 5 minutes, the aqueous layer was frozen in a mixture of acetone and dry ice and the organic layer was transferred to another extraction tube. 50 μL of 2,2-dimethoxypropane was added and the samples were evaporated to dryness under nitrogen at approximately 30° C. The residue was re-dissolved in 300 μL of a mixture of heptane and acetone (90:10, v/v) [or a mixture of heptane and THF (80:20, v/v)] by vortex mixing for 60 seconds. The sample was transferred to an injection vial and an aliquot of 60 μL of the sample was injected into the chromatographic system for analysis with the following chromatographic conditions:
Mobile phase: (Isocratic elution) heptane/acetone/methanol (80:19:1, v/v/v)
Make-up solvent: acetonitrile/acetone/methanol/formic acid (40:60:1:1, v/v/v/v)
Column temperature: −1° C.
Flow rate: 1.00 mL/minute (including 0.750 mL/min mobile phase and 0.250 mL/min make-up solvent, completely transferred to detector)
Injection volume: 60 μL
Auto-sampler temperature: 3° C.

### Example 8

### Combination Therapy with VX-950

A V-950 combination therapy was conducted to determine the safety of VX-950 and its antiviral response. Specifically, this study included 12 treatment-naïve patients infected with genotype 1 HCV. All patients received VX-950 (750 mg q8h), Peg-IFN alfa-2a (“PEG-IFN”, 180 μg weekly), and RBV (1000 or 1200 mg daily), for a period of 28 days. At the completion of the 28 days, patients began off-study follow-on therapy with Peg-IFNα-2a and RBV under the clinical care of their physicians. Additional HCV RNA assessments were performed at the discretion of the treating physicians during the Peg-IFN-2a/RBV therapy. These included assessments at 4, 8, 14 weeks post-study treatment and later timepoints.

The results show that the VX-950/Peg-IFN/RBV combination was well tolerated in the 28-day study, with no serious adverse events. The observed adverse event profile was consistent with the profile commonly seen with the Peg-IFN/RBV combination therapy. All patients demonstrated a response to the study drug regimen, indicating a rapid and substantial antiviral effect of VX-950. Specifically, 2 patients reached undetectable (<10 IU/mL, Roche Taqman® Assay) levels of plasma HCV RNA within 8 days from the start of dosing, and all patients had undetectable HCV RNA at the end of the 28-day study dosing period. At 12 weeks of follow-on therapy (with Peg-IFN/RBV) after completing the 28-day study dosing, the HCV RNA levels remain undetectable in 11 patients. All patients continued on Peg-IFN/RBV therapy, and were followed for response in accordance with standard practice. Seven patients received a total of 48 weeks of treatment and achieved sustained viral response (SVR). One patient received Peg-IFN/RBV for only 18 weeks (total treatment 22 weeks) before discontinuing, but also achieved SVR. Two patients had viral breakthroughs at 12 weeks and 24 weeks of treatment and two patients have been lost to follow up. In total, 8 out of 10 patients from whom the results were available, achieved SVR. The side effect profile observed during the post-study dosing was consistent with the expected profile of Peg-IFN/RBV therapy.

The observation that SVR was achieved in eight patients, including 1 who completed only 22 weeks of treatment, indicates that VX-950-based regimens may allow increased SVR rates as compared to current therapies.

### Compared to Current Treatment

The current treatment for patients with genotype 1 chronic HCV usually consists of 48 weeks of therapy with only pegylated interferon-alfa-2a/2b (Peg-IFN-2a) and RBV, which results in SVR in only about 50% of patients with genotype-1 HCV and the patients generally show poor tolerability of the treatments.

### Example 9

### Phase 1b Studies

VX-950 had rapid and profound antiviral activity as a single agent or in combination with Peg-IFN-2a, and was well tolerated for 14 days. This study was designed to provide information on the kinetics of HCV following treatment with VX-950 and Peg-IFN-2a administered over 14 days.

This study randomized twenty treatment-naïve patients with chronic genotype 1 hepatitis C infection to three dosing arms (Table 1). At the completion of the 14-day study, 19 of 20 patients chose to begin Peg-IFN-2a/RBV, starting within 5 days of completing the 14-day dosing period; as the other one declined to take the combination of Peg-IRN-2a and RBV. Clinic visits were conducted at the discretion of the investigators, after completion of the 1-week and 12-week study-mandated follow-up visits. Nineteen patients have been followed through 24 weeks after the completion of the study dosing. After discussion with the treating physicians, ten (4 in VX-950 and 6 in VX-950/Peg-IFN-2a) patients stopped Peg-IFN-2a/RBV treatment at 24 weeks. The current disposition of the patients is presented in Table 1 below.

TABLE 1
DISPOSITION OF PATIENTS

| | | | Tela- | |
|---|---|---|---|---|
| | Placebo + | Tela- | previr + | |
| | PegIFN-2a | previr | PegIRN-2a | Total |
| | N | N | N | N |
| Enrolled | 4 | 8 | 8 | 20 |
| Dosed | 4 | 8 | 8 | 20 |
| Completed 2 Weeks of | 4 | 8 | 8 | 20 |
| Treatment | | | | |

Off Study Treatment (PegIFN-2a/RBV)

| | | | | |
|---|---|---|---|---|
| Completed 1-Week Safety | 4 | 8 | 8 | 20 |
| Follow-up On-Study | | | | |
| Completed 12-Week Antiviral | 4 | 7 | 8 | 19 |
| Follow-up On-Study | | | | |
| Completed 24-Week Antiviral | 4 | 7 | 8 | 19 |
| Follow-up Off-Study | | | | |
| Peg-IFN-2a/RBV | 0 | 4 | 6 | 10 |
| Discontinuation at 24 weeks | | | | |
| due to decision of the patients | | | | |

At the last off-study follow-up day (12 weeks after the last on-study follow-up), HCV RNA levels were undetectable in all patients who continued with Peg-IFN-2a/RBV, initially randomized in the VX-950 alone and VX-950/Peg-IFN-2a groups. The data are provided below in Table 2.

TABLE 2
Undetectable HCV RNA by groups during the post study-treatment period

| | HCV RNA below | | HCV RNA below | | HCV RNA below |
|---|---|---|---|---|---|
| | limit of quantitation^{a} | | limit of detection^{a} | | Undetectable^{b} |
| | (30 IU/mL) | | (10 IU/mL) | | n |
| | n | | n | | Peg-IFN-2a/RBV Off-Study |
| | Peg-IFN-2a/RBV On-Study | | Peg-IFN-2a/RBV On-Study | | 24-week F/U (12 weeks after |
| | 1-week F/U | 12-week F/U | 1-week F/U | 12-week F/U | last on-study follow-up) |
| VX-950 | 3 | 6 | 1 | 5 | 7 |
| (N-7) | | | | | |
| VX-950/Peg- | 6 | 8 | 3 | 8 | 8 |
| IFN-2a (N-8) | | | | | |
| Peg-IFN-2a | 0 | 3 | 0 | 1 | 3 |
| (N-4) | | | | | |

^{a}COBAS Taqman HCV RNA assay. Roche Molecular Diagnostics
^{b}Taqman HCV RNA assay (15 IU/mL) and 5 IU/mL): off-study

As shown below in Table 3, of the 10 patients who stopped post-study Peg-IFN-2a/RBV treatment after 24 weeks total treatment, 2 of 4 patients who originally received VX-950 alone demonstrated undetectable plasma HCV RNA level at 12 weeks follow-up after stopping Peg-IFN-2a; 5 of 6 patients who originally received VX-950/Peg-IFN-2a demonstrated undetectable plasma HCV RNA level at 12 weeks follow-up after stopping Peg-IFN-2a

TABLE 3
Undetectable HCV RNA by groups following
Peg-IFN-2a/RBV discontinuation

| | | Patients who | Undetectable HCV |
|---|---|---|---|
| | Undetectable HCV | stopped peg- | RNA at 12 weeks |
| | RNA at 24-week | IFN-2a/ | follow-up after |
| | off study peg-IGN- | RBV at Week | stopping peg-IFN- |
| | 2a/RBV treatment | 24 | 2a/RBV |
| | N | n/N | n/N |
| VX-950 | 7* | 4/7 | 2/4 |
| (N-7) | | | |
| VX-950/ | 8 | 6/8 | 5/6 |
| Peg- | | | |
| IFN-2a | | | |
| (N-8) | | | |
| Peg- | 3 | 0/4 | N/A |
| IFN-2a | | | |
| (N-4) | | | |

*One patient declined Peg-IFN-2a/RBV.

At 24-week off-study follow-up, all the patients who were initially randomized in VX-950 groups and continued with Peg-IFN-2a/RBV, maintained undetectable HCV RNA. The early (12-week) post-treatment (Peg-IFN-2a/RBV) follow-up viral load data were consistent with models, suggesting the required duration to achieve SVR was related to the kinetics of early viral clearance. SVR was achieved in 10 of 15 patients who received 14 days of therapy of VX-950 optionally in combination with Peg-INF, followed by Peg-INF/RBV for an additional 22 or 46 weeks.

At week 12, all 8 patients who received an initial combination of VX-950 with PEG-IFN and 5 of 7 patients who received VX-950 alone had undetectable HCV RNA. At week 24, all 15 patients who received VX-950 had undetectable HCV RNA. 10 patients (6 of 8 VX-950 with PEG-IFN and 4 of 7 VX-950 alone) decided to stop PEG-IFN/RBV at week 24 and 5 patients continued treatment of PEG-IFN/RBV for a total of 48 weeks. All groups were followed for an additional 24 weeks. In patients who received at least 14 days of VX-950 (alone or in combination with PEG-IFN) before starting the PEG-IFN with RBV, 7 of 10 patients treated for 24 weeks and 3 of 5 patients treated for 48 weeks achieved SVR.

All of the documents cited herein, are incorporated herein by reference.

### Other Embodiments

While a number of embodiments and examples of this invention are described herein, it is apparent that these embodiments and examples may be altered to provide additional embodiments and examples which utilize the pharmaceutical formulations and drug regimens of this invention. Therefore, it will be appreciated that the scope of this invention is to be defined by the appended claims rather than by the specific embodiments that have been represented by way of example above.

What is claimed is:

1. A therapeutic regimen comprising administering to a patient interferon and ribavirin with VX-950 in an initial phase and administering interferon and ribavirin over a secondary phase, wherein the secondary phase occurs after the initial phase.

2. The therapeutic regimen of claim 1, wherein VX-950 is administered for about 8-12 weeks.

3. The therapeutic regimen of claim 1, wherein VX-950 is administered for about 12 weeks.

4. The therapeutic regimen of claim 1, wherein VX-950 is administered for less than about 12 weeks.

5. The therapeutic regimen of claim 1, wherein VX-950 is administered for about 10 weeks.

6. The therapeutic regimen of claim 1, wherein VX-950 is administered for about 8 weeks.

7. The therapeutic regimen of claim 1, wherein VX-950 is administered for less than about 8 weeks.

8. The therapeutic regimen of claim 1, wherein the interferon is pegylated.

9. The therapeutic regimen of claim 8, wherein the pegylated interferon is interferon-alfa 2a.

10. The therapeutic regimen of claim 8, wherein the pegylated interferon is interferon-alfa 2b.

11. The therapeutic regimen of claim 1, wherein VX-950 is administered in an amount of about 100 mg to about 1500 mg.

12. The therapeutic regimen of claim 11, wherein VX-950 is administered in an amount of about 300 mg to about 1250 mg.

13. The therapeutic regimen of claim 12, wherein VX-950 is administered in an amount of about 450 mg.

14. The therapeutic regimen of claim 12, wherein VX-950 is administered in an amount of about 750 mg.

15. The therapeutic regimen of claim 12, wherein VX-950 is administered in an amount of about 1200 mg.

16. The therapeutic regimen of claim 1, wherein VX-950 is administered twice per day.

17. The therapeutic regimen of claim 16, wherein VX-950 is administered every 12 hours.

18. The therapeutic regimen of claim 1, wherein VX-950 is administered three times per day.

19. The therapeutic regimen of claim 18, wherein VX-950 is administered every 8 hours.

20. A therapeutic regimen comprising administering VX-950 in an amount of about 100 mg to about 1500 mg, wherein this amount of VX-950 is administered for about 12 weeks or less.

21. The therapeutic regimen of claim 20, wherein VX-950 is administered for about 8-12 weeks.

22. The therapeutic regimen of claim 20, wherein VX-950 is administered for about 12 weeks.

23. The therapeutic regimen of claim 20, wherein VX-950 is administered for less than about 12 weeks.

24. The therapeutic regimen of claim 20, wherein VX-950 is administered for about 10 weeks.

25. The therapeutic regimen of claim 20, wherein VX-950 is administered for about 8 weeks.

26. The therapeutic regimen of claim 20, wherein VX-950 is administered for less than about 8 weeks.

27. The therapeutic regimen of claim 20, further comprising administering interferon.

28. The therapeutic regimen of claim 27, wherein the interferon is pegylated.

29. The therapeutic regimen of claim 28, wherein the pegylated interferon is interferon-alfa 2a.

30. The therapeutic regimen of claim 28, wherein the pegylated interferon is interferon-alfa 2b.

31. The therapeutic regimen of claim 20, wherein VX-950 is administered in an amount of about 300 mg to about 1250 mg.

32. The therapeutic regimen of claim 31, wherein VX-950 is administered in an amount of about 450 milligrams.

33. The therapeutic regimen of claim 31, wherein VX-950 is administered in an amount of about 750 milligrams.

34. The therapeutic regimen of claim 31, wherein VX-950 is administered in an amount of about 1200 milligrams.

35. The therapeutic regimen of claim 20, wherein VX-950 is administered twice per day.

36. The therapeutic regimen of claim 35, wherein VX-950 is administered every 12 hours.

37. The therapeutic regimen of claim 20, wherein VX-950 is administered three times per day.

38. The therapeutic regimen of claim 37, wherein VX-950 is administered every 8 hours.

39. The therapeutic regimen of claim 20, further comprising administering ribavirin.

40. The therapeutic regimen of claim 1, further comprising administering to the patient an immunomodulatory agent, an antiviral agent, another inhibitor of HCV NS3/4A protease, an inhibitor of a target in the HCV life cycle other than NS3/4A protease, an inhibitor of internal ribosome entry, a broad-spectrum viral inhibitor, another cytochrome P-450 inhibitor, an inhibitor of viral cellular entry, or a combination thereof.

41. The therapeutic regimen of claim 20, further comprising administering to the patient an immunomodulatory agent, an antiviral agent, another inhibitor of HCV NS3/4A protease, an inhibitor of a target in the HCV life cycle other than NS3/4A protease, an inhibitor of internal ribosome entry, a broad-spectrum viral inhibitor, another cytochrome P-450 inhibitor, an inhibitor of viral cellular entry, or a combination thereof.
